(19) United States
(12) Patent Application Publication
(10) Pub. No.: US 2013/0218234 A1
(43) Pub. Date: Aug. 22, 2013

(54) APPARATUS AND METHOD FOR RAPID SUPPRESSION OF NEUROPATHIC, ONCOLOGICAL, AND PAEDIATRIC PAIN

(71) Applicant: Giuseppe Marineo, Roma (IT)

(72) Inventor: Giuseppe Marineo, Roma (IT)

(21) Appl. No.: 13/882,974

(22) PCT Filed: Nov. 16, 2010
(86) PCT No.: PCT/IT2010/000457

§ 371 Date: May 1, 2013

(51) Int. Cl. A61N 1/36

(52) U.S. Cl. CPC A61N 1/36071
USPC 607/46

(57) ABSTRACT

The present invention relates to an apparatus and to a method for rapid suppression of acute and chronic pain, which can be used also in the paediatric field or with particular forms of pain such as chemotherapy-induced peripheral neuropathy (CIPN) and neuralgias that affect the eye bulb, and is in general particularly useful and effective in regard to pains of high degree and/or resistant to other analgesics, such as opiates or other forms of conventional electro-analgesia such as transcutaneous electrical nerve stimulators (TENS) and implanted stimulators. According to the present invention, strings of synthetic “non-pain” information of considerable effectiveness are generated, such as to enable a high reproducibility of the clinical result. The synthesis is made by combining new geometries of waveforms and new modulations in complex sequences, perceived instantaneously as “self” and as “non-pain” by the CN. S.

The present invention relates to an apparatus and to a method for rapid suppression of acute and chronic pain, which is particularly useful and effective in regard to pains of high degree and/or resistant to other analgesics such as opiates, or to conventional electro-analgesia performed with transcutaneous electrical nerve stimulators (TENS) or implanted stimulators.

Pain therapy by means of electrostimulation is performed with equipment that normally produces wave trains with a frequency of between 5 and 100 Hz, with variable duty cycle, at times implementing automatic scans in frequency and amplitude. This equipment is conventionally referred to as TENS, when it is used in a non-invasive way with surface electrodes, or as implanted electrostimulators, when it is invasive. This kind of electro-analgesia, with reference to the accredited scientific literature, functions only in some types of pain, prevalently muscular pain, but hardly ever functions or functions with largely unsatisfactory and unforeseeable results in chronic pain of high degree of a neuropathic and oncological type and in pain non-responsive to morphine and/or derivatives.

These electrostimulations moreover have a basis of scientific and technological development that is substantially heuristic. There does not exist, in fact, in the scientific literature, a commonly accepted explanation of the biological mechanisms of the analgesic effect that in some cases is produced. One of the theories that still today is considered as plausible is that the electrical stimulus favours production of endorphins, and these in turn are responsible of analgesia. In actual fact, targeted clinical research that has been published has called this tentative explanation into considerable doubt.

More accredited and rational is the explanation of the above mechanism according to the “gate-control theory”. The hypothesis hence is that these electrostimulations have the function of inhibiting the transmission of the painful stimulus, carrying out a blockage of nervous conduction of an electrical type. According to the gate-control theory, this is obtained by stimulating the A-Beta fibres, hence those responsible for tactile conduction. The differential effect between the activity of the A-Beta fibres and that of the C fibres, the latter being specifically responsible for the transmission of pain, would enable modulation of the perception of pain, reducing it when the activity of the A-Beta fibres prevails, and increasing it when the activity of the C fibres prevails. Since 1965 up to the present day the gate-control theory has received numerous scientific confirmations based upon published experimental data, and has been the guide for the development of different therapies used in the control of pain.

In line with this theory conventional electro-analgesia uses pulses of very short duration, generally comprised between 50 and 250 μs. The reason for this choice is that the A-Beta fibres are fast-conduction (myelinic) fibres, and can respond to very short stimuli. The C fibres are, instead, slow-conduction (amyelinic) fibres, and to be excited require longer stimuli, of the order of milliseconds. In brief, conventional electro-analgesia becomes selective in regard to A-Beta fibres by choosing very short pulses of appropriate duration. The known limit of the gate-control theory is that it is able to provide a good explanation of acute pain, where the cause-effect relationships are linear, whereas it is unable to explain equally effectively chronic pain, where the cause-effect relationships lose linearity and assume characteristics that are so particular as to have introduced in the scientific field the need to classify chronic pain as an independent pathological condition, and no longer a physiological reaction of a protective type.

For this reason, underlying the present invention is a theoretical model of pain developed propaedeutically by the author and forming the subject of scientific publications, which provides a rational explanation of chronic pain from a cybernetic standpoint, completely disregarding the gate-control theory. In particular, whereas the gate-control theory excludes the possibility of exciting the C fibres, since the differential effect would be disadvantageous, the present invention uses them as primary vehicle for inducing analgesia, without blocking conduction thereof, thus completely departing from the traditional technology of electro-analgesia and the gate-control theory. It should be added that a simple electrical stimulus that is to excite the C fibres typically produces pain. To obtain analgesia by exciting the C fibres, it is necessary to convert the electrical stimulus into “non-pain” information, as envisaged by the theory developed by the author of the present invention, which renders possible application thereof to the clinical sector.

The aim of the present invention is to tackle the problem of oncological pain and of chronic pain of high intensity that is not responsive to any other protocol treatment, with extension to the paediatric field, and to some particular types of neuropathic pain, such as chemotherapy-induced peripheral neuropathy (CIPN), which require certain some important innovations that will be described more fully hereinafter. The theoretical studies of the author have led to the technological development of an “artificial neuron” capable of generating strings of “non-pain” information. This artificial bioinformation, by modulating appropriate electropotentials sent into the nervous network via surface electrodes, superposes on the endogenous information that encodes the pain, thus obtaining a powerful analgesic effect that is practically instantaneous and independent of the intensity of pain and of the specific pathological condition.

A series of prior patents (IT-A-1324899, WO-A1-2009/037721) filed in the name of the present applicant, have begun introduce the concepts of the so-called “scrambler therapy”, based upon the concept of synthetic “non-pain” information for therapeutic purposes. The prior patents regard the progressive evolution of an apparatus designed to implement an “artificial neuron” that is increasingly sophisticated and optimized as regards its applications and clinical results.

In the progress of knowledge and of the new clinical applications, some needs for updating have emerged, said updating forming the subject of the present patent, which solves the problems listed below.

1) Application in the Paediatric Field

Application in the paediatric field of the techniques according to the above prior patents was rendered difficult by a particular sort of “pricking” sensation that the patient felt during the step of regulation of intensity of the stimulus, a sensation that disappeared once regulation was performed. Technically, this sensation was due to the fact that the sub-stimulation typical of the regulation step, during the modulation peaks, could excite the A-Delta fibres, arousing the more or less intense characteristic pricking sensation. Whereas in an adult duly pre-warned of this temporary sensation the discomfort was tolerated, in a child the fear generated by this unpleasant sensation could prevent conclusion of the step of regulation that would have eliminated it and would have produced the required analgesia. In brief, the fact of not eliminating this sensation would have made it difficult or impossible to treat smaller children or ones emotively more sensitive.

2) Optimized Application in Chemotherapy-Induced Peripheral Neuropathy (CIPN)

CIPN, which is a serious form of neuropathy consequent upon chemotherapy, prevalently affects the lower and upper limbs, and profoundly alters the patient's perceptions also in the areas not affected by pain. In this case, it has been necessary to modify some characteristics of the synthetic “non-pain” information in order to simplify application of the technique to this type of patients, where the particular localization and extension of pain makes it difficult to position the electrodes and adjust the level of stimulation to achieve the optimal level.

3) Application in Chronic Neuropathic Pain Localized in the Eye Bulb

Also in this case, the particular sensitivity of the area affected has required substantial improvements in terms of tolerability of the stimulus in order to increase the possibilities of patient compliance. This particular form of pain, on account to the devastating effects that it has on the quality of life, statistically leads to a higher incidence of suicide as compared to other forms of chronic pain.

4) Need for Compatibility with Different Types of Electrodes that Derive from the Introduction on the Market of Variant Types of Electrodes Having Properties that are Very Different from Those of Electrodes Used During Previous Development Steps

Right from the outset the technique underlying the present invention was developed so as to ensure compatibility with disposable ECG electrodes. Over time the technology of production of these electrodes has changed, thus posing significant problems of electrical compatibility. It has thus been necessary to find innovative solutions to widen further the range of operativeness of the automatic-regulation systems, and thus overcome the above problems, which are increasingly felt as a result of the changes in the market of ECG electrodes. It is to be noted that these electrodes have not been produced for electrostimulation, but for analyses of the ECG biopotentials. Since the devices that carry out these analyses are provided with high-impedance differential inputs and do not produce electrical currents able to modify the electrical properties of the conductor over time, they are not affected by the change of technology of production.

The present invention solves the problems set forth above by introducing the technological innovations described more fully hereinafter, to provide an apparatus for rapid pain suppression, a method for its operation, a definition of one or more waveforms to be produced and used for generating an electrical signal in a therapy for rapid pain suppression, and a method for generating an electrical signal to be used in a therapy for rapid pain suppression, as defined in the respective independent claims.

Secondary characteristics of the present invention are, instead, defined in the respective dependent claims.

The present invention, by overcoming the aforesaid problems of the known art, leads to numerous and evident advantages.

The main advantage lies in the fact that the result of this process of hardware and software innovation is the generation and control of strings of synthetic “non-pain” information of considerable effectiveness that are more complex than those of the prior art but enable a greater reproducibility of the clinical result when the latter depends upon human variables or variables of the pathological condition that assume the form of specific sensitivity of the patient, this rendering the innovative apparatus markedly compatible with the problems inherent in use on children and on persons affected by CIPN or by pain localized in particularly sensitive areas, such as the eyes, said innovative apparatus being moreover interfaceable with a wide range of commercially available disposable electrodes, with electrical characteristics markedly different according to the type of production.

This further work of widening the variability and tolerability of the synthetic “non-pain” information has involved the need to use new geometries of waveforms (which as a whole constitute a base, comparable to letters of the alphabet, of the wider variability synthetic “non-pain” information), modifications to the control algorithm for the definitive assemblage in dynamic strings, i.e., in information more complex than an individual waveform, new circuits for dynamic regulation of the envelopes and of the feedbacks necessary for proper operation of the device, which are all elements that, in their indispensable synergies, achieve in an optimal way the clinical effectiveness and tolerability of the technique according to the present invention in chronic pain that was previously considered untreatable.

Other advantages, characteristics, and modalities of use of the present invention will emerge clearly from the ensuing detailed description of an embodiment thereof, provided by way of non-limiting example. Reference will be made to the figures of the attached plates of drawings, wherein:

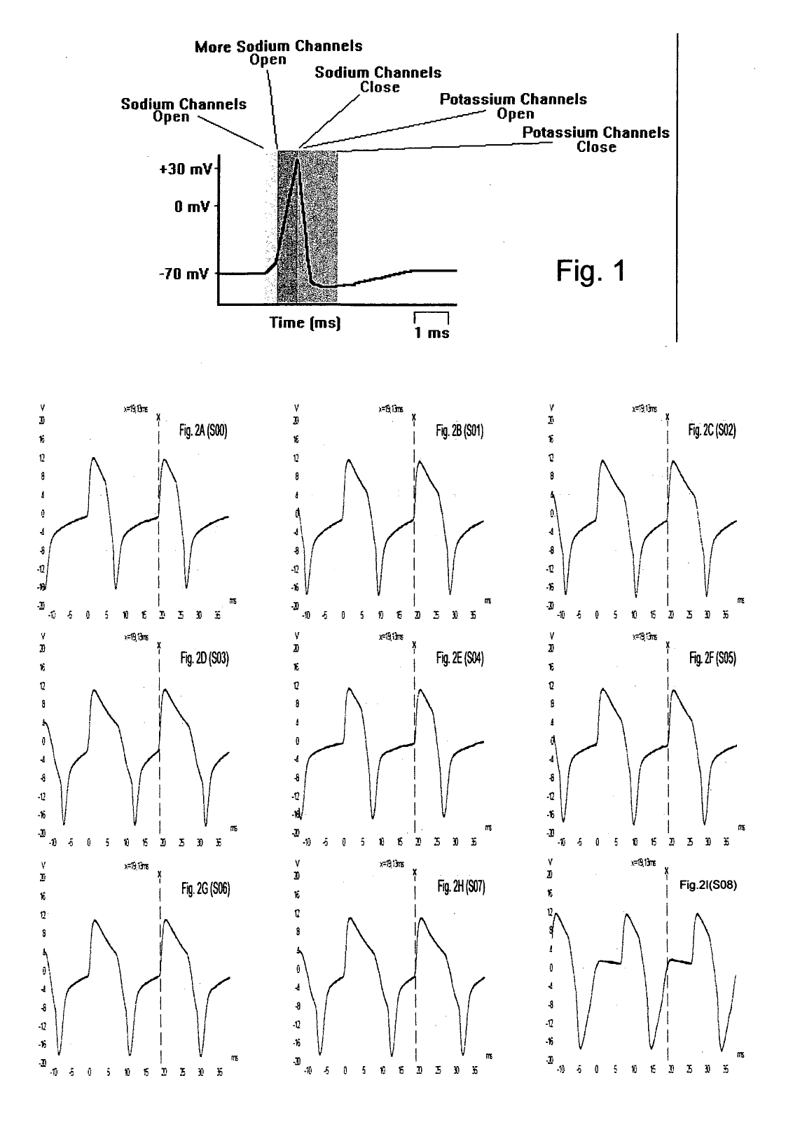

FIG. 1 shows a typical action potential produced by human nerve cells;

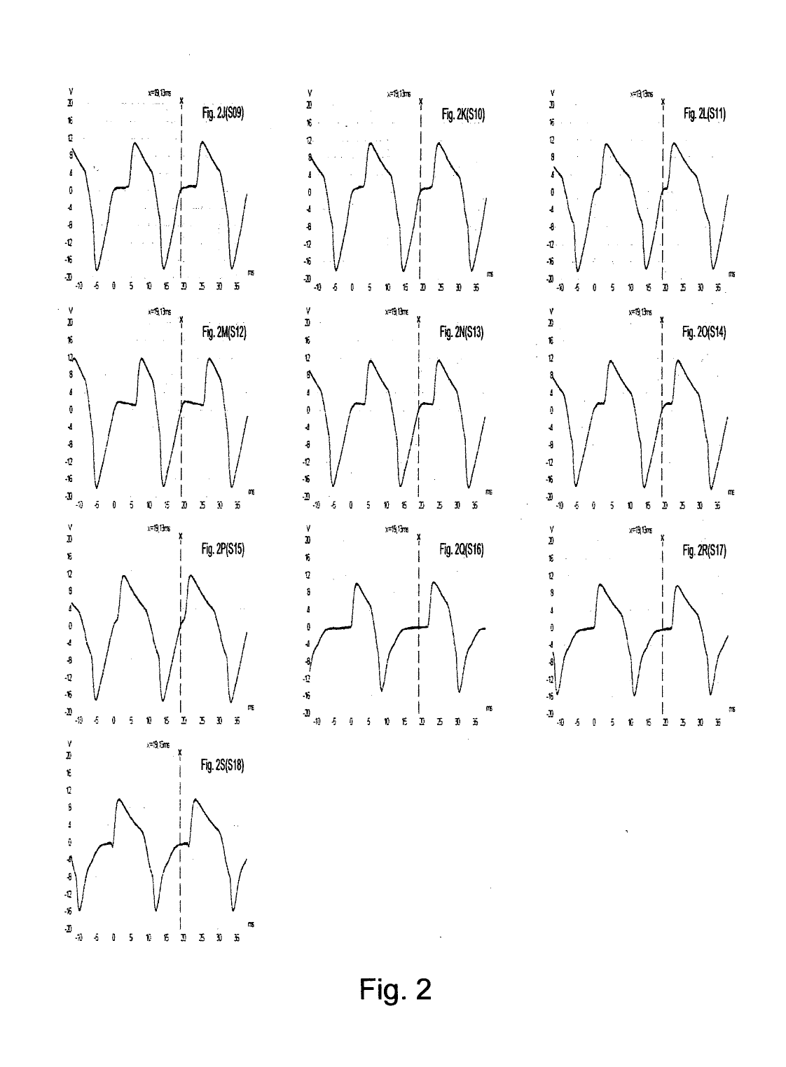

FIGS. 2A to 2S are graphs representing the time plot of the nineteen output waveforms processed from the digital primitives described numerically according to the present invention;

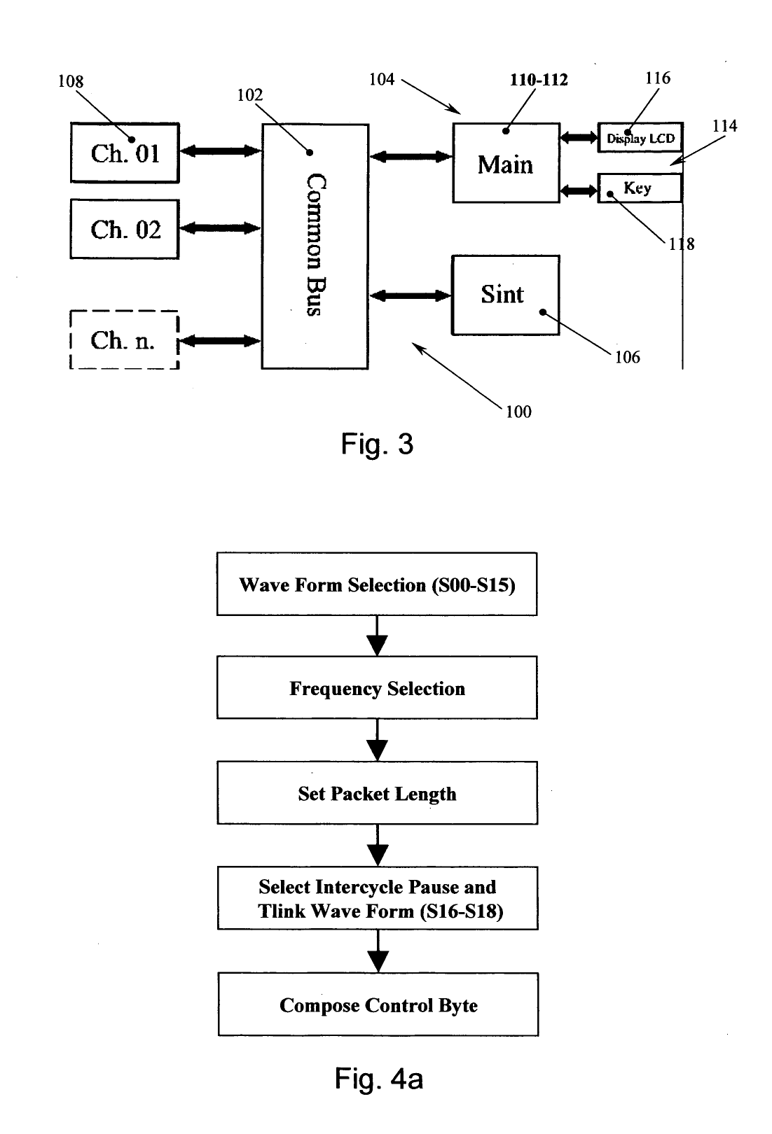

FIG. 3 is a block diagram of an apparatus according to the present invention;

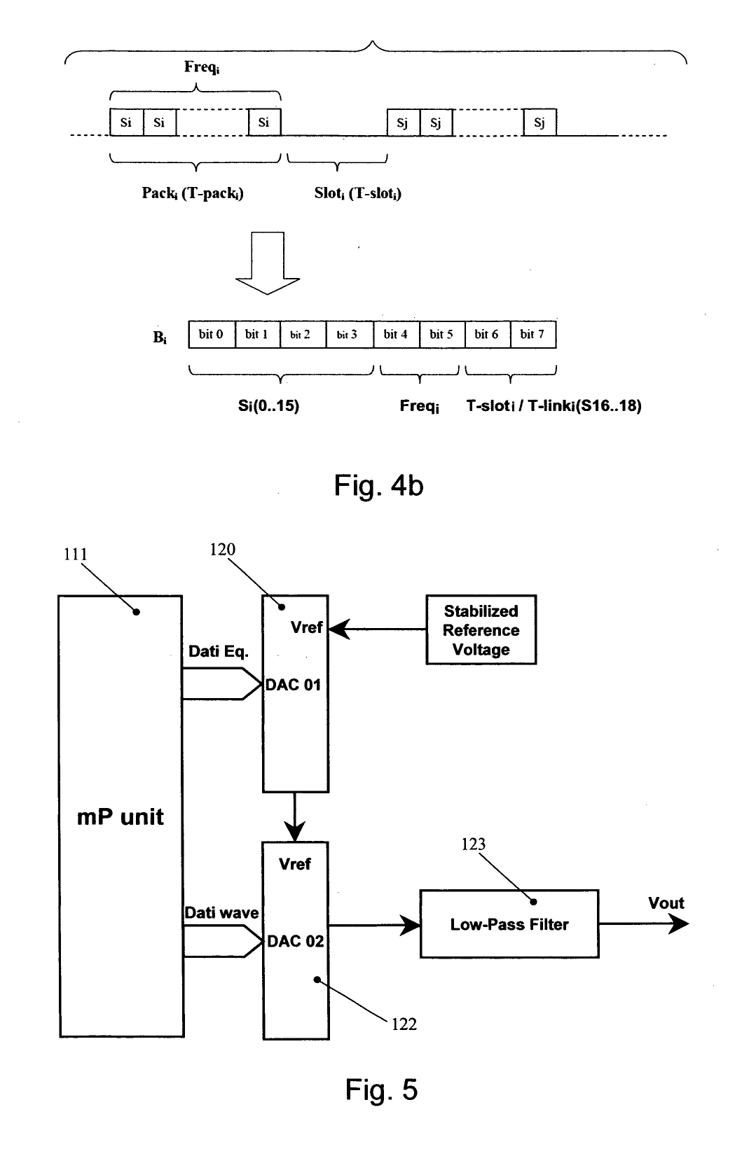

FIG. 4A is a flowchart that represents schematically an algorithm for controlling the synthesis according to the present invention;

FIG. 4B is a schematic illustration of the result of the algorithm, in terms of sequence of data S and of control bytes Si;

FIG. 5 is a circuit diagram of a synthesizer module according to the present invention;

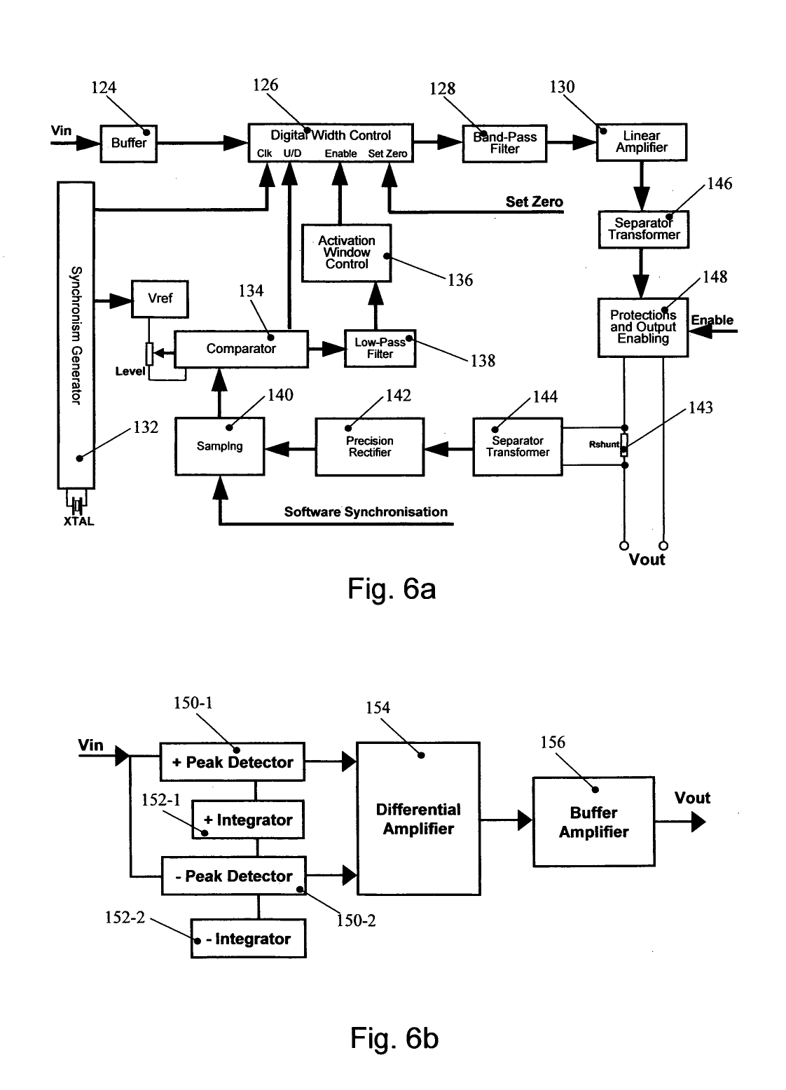

FIG. 6 is a block diagram of a channel module according to the present invention;

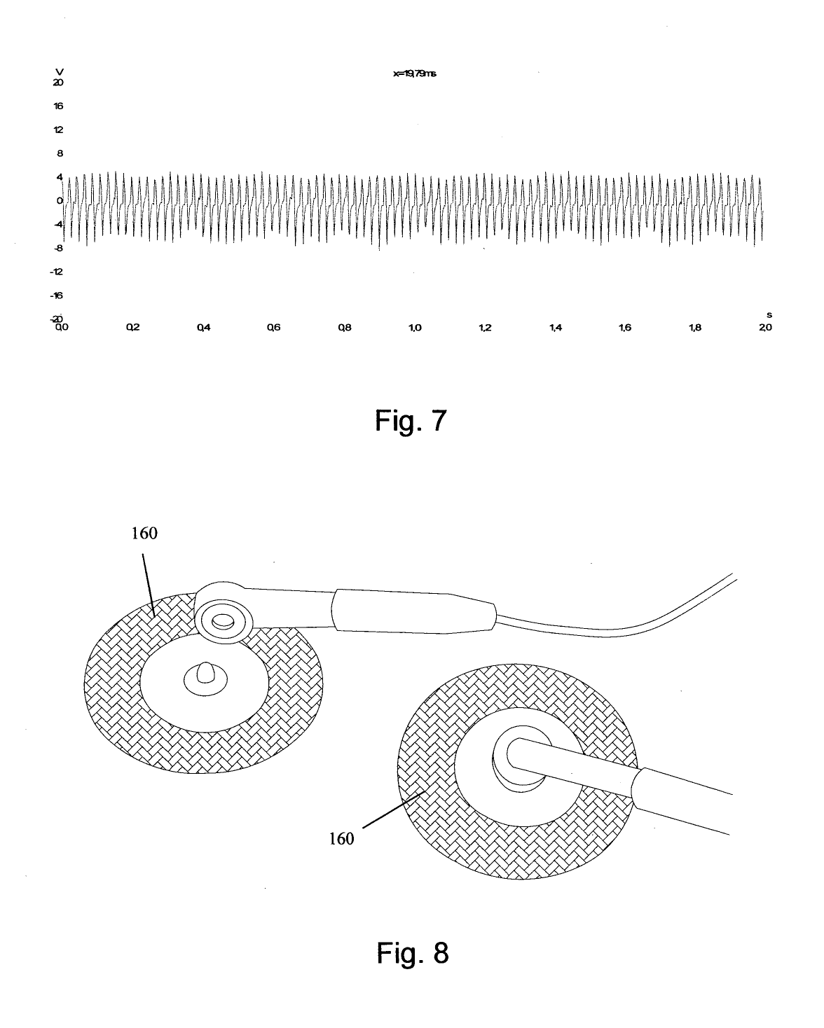

FIG. 7 shows an example of the modulation on a portion of one of the packets that constitute the string;

FIG. 8 is a view of a pair of different electrodes to be used in implementing the present invention;

FIGS. 10 to 12 show examples of positioning or arrangement of the electrodes on the body of a patient, according to the methodology of the present invention.

The present invention will be described in what follows with reference to the aforementioned figures.

The present invention is based upon the theoretical observation outlined below. As is known, the “pain system” is characterized by a high information content, which in itself constitutes its essence. The datum of interest, which is here taken into account, is the central role of the control of the “pain” information in regard of the chemico-structural variations of the pain system as a whole, and in its variegated clinical manifestations.

According to the present invention, it is deemed possible to control the lower levels of complexity of the pain system, i.e., the biochemical levels, by manipulating at higher levels of complexity (the levels of the bioelectrical signals generated by the nerve cells) exclusively the associated “information” variable, which at these levels having emerging properties can be treated easily by encoding electrical potentials in syntheses of waveforms having variable geometry and dynamic structure of assemblage, with information function analogous to that proper to nerve cells.

The present invention hence consists in the possibility of manipulating conveniently the endogenous “pain” information replacing it with a synthetic information, recognized as “self” by the organism, and perceived as “non-pain”. By “non-pain” is meant a series of substitutive sensations that, during the treatment, the patient perceives instead of the pain, said sensations being attributable to the pre-pain activity of the polymodal receptors that in the nociceptive system belong to the C fibres.

From an information standpoint the geometry of the individual base waveforms (see FIGS. 2A to 2S) basically represents an alphabet of letters that, assembled dynamically in strings of variable length and content, constructs the equivalent of a complex of synthetic “non-pain” information that the nervous system recognizes as “self” once said information has been transferred into the nervous system by the surface receptors.

This synthetic information is able to overmodulate the endogenous pain information, obtaining as clinical effect the immediate disappearance of the perception of pain, irrespective of its intensity, chronicity, benign or oncological nature, presence of neuropathic damage, resistance to opiates or other forms of electro-analgesia.

From what has been set forth above, it is possible to infer that it has been essential, in order to obtain the desired result, to select in an extremely precise and targeted way the geometry of the waveforms used. It is likewise easy to imagine that the possibilities of synthesis are in principle substantially infinite, since, irrespective of logico-analytical evaluations of a propaedeutic nature, in biology there do not exist certain and immutable rules and linear behaviours. Hence, the selection used considered optimal has been performed starting from an analytical structure subsequently improved through numerous clinical studies and experiments purposely conducted over a broad statistical base.

Consequently, since they are synthesized waveforms and cannot be described in a simple way through mathematical models, described hereinafter is a set of primitive waveforms (S00-S18), i.e., waveforms in their first formation by digital synthesis, which subsequently will undergo further processing, said primitive waveforms being used on the basis of respective first parameters Vi that identify them, in particular on the basis of their respective values of amplitude, which are used for the synthesis. Each primitive waveform has a periodic and predetermined time plot. This fact, in association with a very precise possible frequency that will be indicated—and provided that the steps of digital-to-analog conversion necessary and the respective associated values of D/A conversion (vectors Vi) are known—, enables an exact reconstruction of the waveform.

Appearing in the ensuing Table 1 are the values of amplitude, expressed in the hexadecimal system, used for the synthesis of the primitive waveforms. According to the preferred embodiment of the present invention, each individual primitive waveform Si is represented numerically by a vector Vi of sixty-four 8-bit values.

TABLE 1
Primitive waveform
vector Vi of the values of amplitude (i = 0 . . . 18)
S00

| B6 | FE | FE | FE | FE | FE | FE | FE | FE | FE | FE | FE | FE | FE | FE | FE | EC | BY | C8 |
|---|---|---|---|---|---|---|---|---|---|---|---|---|---|---|---|---|---|---|
| B6 | A4 | 92 | 7F | 00 | 20 | 40 | 60 | 6E | 80 | 80 | 80 | 80 | 80 | 80 | 80 | 80 | 80 | 80 |
| 80 | 80 | 80 | 80 | 80 | 80 | 80 | 80 | 80 | 80 | 80 | 80 | 80 | 80 | 80 | 80 | 80 | 80 | 80 |
| 80 | 80 | 80 | 80 | 80 | 80 | | | | | | | | | | | | | |

S01

| | | | | | | | | | | | | | | | | | | |
|---|---|---|---|---|---|---|---|---|---|---|---|---|---|---|---|---|---|---|
| 81 | B6 | FE | FE | FE | FE | FE | FE | FE | FE | FE | FE | FE | FE | FE | FE | FE | FE | FE |
| FE | FE | FE | FA | EC | DE | D0 | C2 | B4 | A6 | 9A | 8E | 00 | 20 | 40 | 60 | 6E | 80 | 80 |
| 80 | 80 | 80 | 80 | 80 | 80 | 80 | 80 | 80 | 80 | 80 | 80 | 80 | 80 | 80 | 80 | 80 | 80 | 80 |
| 80 | 80 | 80 | 80 | 80 | 80 | | | | | | | | | | | | | |

S02

| | | | | | | | | | | | | | | | | | | |
|---|---|---|---|---|---|---|---|---|---|---|---|---|---|---|---|---|---|---|
| 81 | AA | D4 | FE | FE | FE | FE | FE | FE | FE | FE | FE | FE | FE | FE | FE | FE | FE | FE |
| FE | FE | FE | FE | FE | FE | FE | F0 | E2 | D4 | C6 | B8 | AA | 9C | 8E | 80 | 00 | 20 | 40 |
| 60 | 6E | 80 | 80 | 80 | 80 | 80 | 80 | 80 | 80 | 80 | 80 | 80 | 80 | 80 | 80 | 80 | 80 | 80 |
| 80 | 80 | 80 | 80 | 80 | 80 | 80 | | | | | | | | | | | | |

S03

| | | | | | | | | | | | | | | | | | | |
|---|---|---|---|---|---|---|---|---|---|---|---|---|---|---|---|---|---|---|
| 81 | AA | D4 | FE | FE | FE | FE | FE | FE | FE | FE | FE | FE | FE | FE | FE | FE | FE | FE |
| FE | FE | FE | FE | FE | FE | FE | FE | FE | FE | F5 | EC | E3 | BY | D1 | C8 | BF | AD | A5 |
| 9B | 92 | 80 | 00 | 20 | 40 | 60 | 6E | 80 | 80 | 80 | 80 | 80 | 80 | 80 | 80 | 80 | 80 | 80 |
| 80 | 80 | 80 | 80 | 80 | 80 | 80 | | | | | | | | | | | | |

S04

| | | | | | | | | | | | | | | | | | | |
|---|---|---|---|---|---|---|---|---|---|---|---|---|---|---|---|---|---|---|
| B6 | FE | FE | FE | FE | FE | FE | FE | FE | FE | FE | FE | FE | FE | FE | FE | EC | BY | C8 |
| B6 | A4 | 92 | 80 | 00 | 10 | 20 | 30 | 40 | 60 | 70 | 80 | 80 | 80 | 80 | 80 | 80 | 80 | 80 |
| 80 | 80 | 80 | 80 | 80 | 80 | 80 | 80 | 80 | 80 | 80 | 80 | 80 | 80 | 80 | 80 | 80 | 80 | 80 |
| 80 | 80 | 80 | 80 | 80 | 80 | 80 | | | | | | | | | | | | |

S05

| | | | | | | | | | | | | | | | | | | |
|---|---|---|---|---|---|---|---|---|---|---|---|---|---|---|---|---|---|---|
| 81 | B6 | FE | FE | FE | FE | FE | FE | FE | FE | FE | FE | FE | FE | FE | FE | FE | FE | FE |
| FE | FE | FE | FA | EC | DE | D0 | C2 | B4 | A6 | 9A | 8E | 00 | 10 | 20 | 30 | 40 | 60 | 70 |
| 80 | 80 | 80 | 80 | 80 | 80 | 80 | 80 | 80 | 80 | 80 | 80 | 80 | 80 | 80 | 80 | 80 | 80 | 80 |
| 80 | 80 | 80 | 80 | 80 | 80 | | | | | | | | | | | | | |

S06

| | | | | | | | | | | | | | | | | | | |
|---|---|---|---|---|---|---|---|---|---|---|---|---|---|---|---|---|---|---|
| 81 | AA | D4 | FE | FE | FE | FE | FE | FE | FE | FE | FE | FE | FE | FE | FE | FE | FE | FE |
| FE | FE | FE | FE | FE | FE | FE | F0 | E2 | D4 | C6 | B8 | AA | 9C | 8E | 80 | 00 | 10 | 20 |
| 30 | 40 | 60 | 70 | 80 | 80 | 80 | 80 | 80 | 80 | 80 | 80 | 80 | 80 | 80 | 80 | 80 | 80 | 80 |
| 80 | 80 | 80 | 80 | 80 | 80 | 80 | | | | | | | | | | | | |

S07

| | | | | | | | | | | | | | | | | | | |
|---|---|---|---|---|---|---|---|---|---|---|---|---|---|---|---|---|---|---|
| 81 | AA | D4 | FE | FE | FE | FE | FE | FE | FE | FE | FE | FE | FE | FE | FE | FE | FE | FE |
| FE | FE | FE | FE | FE | FE | FE | FE | FE | F5 | EC | E3 | BY | D1 | C8 | BF | B6 | AD | A5 |
| 9B | 92 | 80 | 00 | 10 | 20 | 30 | 40 | 60 | 70 | 80 | 80 | 80 | 80 | 80 | 80 | 80 | 80 | 80 |
| 80 | 80 | 80 | 80 | 80 | 80 | 80 | | | | | | | | | | | | |

S08

| | | | | | | | | | | | | | | | | | | |
|---|---|---|---|---|---|---|---|---|---|---|---|---|---|---|---|---|---|---|
| B6 | FE | FE | FE | FE | FE | FE | FE | FE | FE | FE | FE | FE | FE | FE | FE | EC | BY | C8 |
| B6 | A4 | 92 | 80 | 00 | 04 | 08 | 0C | 10 | 16 | 1C | 22 | 28 | 2E | 34 | 3A | 40 | 50 | 60 |
| 70 | 78 | 80 | 80 | 80 | 80 | 80 | 80 | 80 | 80 | 80 | 80 | 80 | 80 | 80 | 80 | 80 | 80 | 80 |
| 80 | 80 | 80 | 80 | 80 | 80 | 80 | | | | | | | | | | | | |

S09

| | | | | | | | | | | | | | | | | | | |
|---|---|---|---|---|---|---|---|---|---|---|---|---|---|---|---|---|---|---|
| 81 | B6 | FE | FE | FE | FE | FE | FE | FE | FE | FE | FE | FE | FE | FE | FE | FE | FE | FE |
| FE | FE | FE | FA | EC | DE | D0 | C2 | B4 | A6 | 9A | 8E | 00 | 04 | 08 | 0C | 10 | 16 | 1C |
| 22 | 28 | 2E | 34 | 3A | 40 | 50 | 60 | 70 | 78 | 80 | 80 | 80 | 80 | 80 | 80 | 80 | 80 | 80 |
| 80 | 80 | 80 | 80 | 80 | 80 | 80 | | | | | | | | | | | | |

S10

| | | | | | | | | | | | | | | | | | | |
|---|---|---|---|---|---|---|---|---|---|---|---|---|---|---|---|---|---|---|
| 81 | AA | D4 | FE | FE | FE | FE | FE | FE | FE | FE | FE | FE | FE | FE | FE | FE | FE | FE |
| FE | FE | FE | FE | FE | FE | FE | F0 | E2 | D4 | C6 | B8 | AA | 9C | 8E | 80 | 00 | 04 | 08 |
| 0C | 10 | 16 | 1C | 22 | 28 | 2E | 34 | 3A | 40 | 50 | 60 | 70 | 78 | 80 | 80 | 80 | 80 | 80 |
| 80 | 80 | 80 | 80 | 80 | 80 | 80 | | | | | | | | | | | | |

S11

| | | | | | | | | | | | | | | | | | | |
|---|---|---|---|---|---|---|---|---|---|---|---|---|---|---|---|---|---|---|
| 81 | AA | D4 | FE | FE | FE | FE | FE | FE | FE | FE | FE | FE | FE | FE | FE | FE | FE | FE |
| FE | FE | FE | FE | FE | FE | FE | FE | FE | F5 | EC | E3 | BY | D1 | C8 | BF | B6 | AD | A5 |
| 9B | 92 | 80 | 00 | 04 | 08 | 0C | 10 | 16 | 1C | 22 | 28 | 2E | 34 | 3A | 40 | 50 | 60 | 70 |
| 78 | 80 | 80 | 80 | 80 | 80 | 80 | | | | | | | | | | | | |

S12

| | | | | | | | | | | | | | | | | | | |
|---|---|---|---|---|---|---|---|---|---|---|---|---|---|---|---|---|---|---|
| B6 | FE | FE | FE | FE | FE | FE | FE | FE | FE | FE | FE | FE | FE | FE | FE | EC | BY | C8 |
| B6 | A4 | 92 | 89 | 00 | 05 | 09 | 0E | 18 | 1E | 20 | 22 | 28 | 2E | 34 | 3A | 40 | 49 | 52 |
| 5B | 64 | 6D | 77 | 7F | 80 | 80 | 80 | 80 | 80 | 80 | 80 | 80 | 80 | 80 | 80 | 80 | 80 | 80 |
| 80 | 80 | 80 | 80 | 80 | 80 | 80 | | | | | | | | | | | | |

S13

| | | | | | | | | | | | | | | | | | | |
|---|---|---|---|---|---|---|---|---|---|---|---|---|---|---|---|---|---|---|
| 81 | B6 | FE | FE | FE | FE | FE | FE | FE | FE | FE | FE | FE | FE | FE | FE | FE | FE | FE |
| FE | FE | FE | FA | EC | DE | D0 | C2 | B4 | A6 | 9A | 8E | 00 | 05 | 09 | 0E | 18 | 1E | 20 |
| 22 | 28 | 2E | 34 | 3A | 40 | 49 | 52 | 5B | 64 | 6D | 77 | 7F | 80 | 80 | 80 | 80 | 80 | 80 |
| 80 | 80 | 80 | 80 | 80 | 80 | 80 | | | | | | | | | | | | |

S14

| | | | | | | | | | | | | | | | | | | |
|---|---|---|---|---|---|---|---|---|---|---|---|---|---|---|---|---|---|---|
| 81 | AA | D4 | FE | FE | FE | FE | FE | FE | FE | FE | FE | FE | FE | FE | FE | FE | FE | FE |
| FE | FE | FE | FE | FE | FE | FE | F0 | E2 | D4 | C6 | B8 | AA | 9C | 8E | 80 | 00 | 05 | 09 |
| 0E | 18 | 1E | 20 | 22 | 28 | 2E | 34 | 3A | 40 | 49 | 52 | 5B | 64 | 6D | 77 | 7F | 80 | 80 |
| 80 | 80 | 80 | 80 | 80 | 80 | 80 | | | | | | | | | | | | |

S15

| | | | | | | | | | | | | | | | | | | |
|---|---|---|---|---|---|---|---|---|---|---|---|---|---|---|---|---|---|---|
| 81 | AA | D4 | FE | FE | FE | FE | FE | FE | FE | FE | FE | FE | FE | FE | FE | FE | FE | FE |
| FE | FE | FE | FE | FE | FE | FE | FE | FE | F5 | EC | E3 | BY | D1 | C8 | BF | B6 | AD | A5 |
| 9B | 92 | 80 | 00 | 05 | 09 | 0E | 18 | 1E | 20 | 22 | 28 | 2E | 34 | 3A | 40 | 49 | 52 | 5B |
| 64 | 6D | 77 | 7F | 80 | 80 | 80 | | | | | | | | | | | | |

S16

| | | | | | | | | | | | | | | | | | | |
|---|---|---|---|---|---|---|---|---|---|---|---|---|---|---|---|---|---|---|
| B6 | FE | FE | FE | FE | FE | FE | FE | FE | FE | FE | FE | FE | FE | FE | FE | EC | BY | C8 |
| B6 | A4 | 92 | 81 | 00 | 11 | 23 | 34 | 3F | 52 | 59 | 61 | 63 | 65 | 67 | 69 | 6B | 70 | 75 |
| 7B | 7D | 80 | 80 | 80 | 80 | 80 | 80 | 80 | 80 | 80 | 80 | 80 | 80 | 80 | 80 | 80 | 80 | 80 |
| 80 | 80 | 80 | 80 | 80 | 80 | 80 | | | | | | | | | | | | |

S17

| | | | | | | | | | | | | | | | | | | |
|---|---|---|---|---|---|---|---|---|---|---|---|---|---|---|---|---|---|---|
| 81 | B6 | FE | FE | FE | FE | FE | FE | FE | FE | FE | FE | FE | FE | FE | FE | FE | FE | FE |
| FE | FE | FE | FA | EC | DE | D0 | C2 | B4 | A6 | 9A | 8E | 00 | 11 | 23 | 34 | 3F | 52 | 59 |
| 61 | 63 | 65 | 67 | 69 | 6B | 70 | 75 | 7B | 7D | 80 | 80 | 80 | 80 | 80 | 80 | 80 | 80 | 80 |
| 80 | 80 | 80 | 80 | 80 | 80 | 80 | | | | | | | | | | | | |

S18

| | | | | | | | | | | | | | | | | | | |
|---|---|---|---|---|---|---|---|---|---|---|---|---|---|---|---|---|---|---|
| 60 | AA | D4 | FE | FE | FE | FE | FE | FE | FE | FE | FE | FE | FE | FE | FE | FE | FE | FE |
| FE | FE | FE | FE | FE | FE | FE | F0 | E2 | D4 | C6 | B8 | AA | 9C | 8E | 81 | 00 | 11 | 23 |
| 34 | 3F | 52 | 59 | 61 | 63 | 65 | 67 | 69 | 6B | 70 | 75 | 7B | 7D | 80 | 80 | 80 | 80 | 80 |
| 80 | 80 | 80 | 80 | 80 | 80 | 80 | | | | | | | | | | | | |

FIGS. 2A to 2S show the plot of the waveforms preferentially selected according to the invention after they have undergone, in addition to the digital synthesis, all the various necessary passages of filtering and further geometrical definition, graphically represented in the geometries numbered from S00 to S18. It is likewise to be understood that also forms that depart from these, for smaller variations in amplitude, in time, or in geometry of one or more samples, could be used for the application of a method such as the one described herein. However, said further waveforms could have lower or even zero effectiveness and/or involve undesirable effects for the patient.

The images shown in said figures have been obtained with an PC oscilloscope (Picoscope 3204), having the following technical characteristics:

Band: 50 MHz

Buffer size: 256 kB

Timebase range: 5 ns/div to 50 s/div

Analog bandwidth: 50 MHz

Precision: 3%

Resolution: 8 bits

Sampling rate: 50 MS/s

An apparatus according to the present invention will now be described.

FIG. 3 shows a block diagram of an apparatus 100 according to the present invention. With reference to the block diagram of FIG. 3, it is possible to identify a common bus “Common Bus” 102 connected to which are the various modules of the apparatus, which will be described in greater detail.

In particular, the modules are: a main management module “Main” 104; a synthesizer module “Synth” 106, which supervises the digital-to-analog conversion of the sequence of the primitive waveforms, as processed by the module Main 104; and one or more output channel modules “Chk” 108, which perform a further analog processing of the signals, before their application to the body of the patient, through electrodes 160 (FIG. 8) arranged in the way described in what follows.

The module Main 104 carries out complete management of the treatment and of the safety devices for the subject undergoing the therapy. Moreover provided is a serial output for possible communications and remote control of the device.

The resident hardware and firmware mainly perform three functions: interfacing with the user; control of the synthesis of the strings of information; and safety for the patient.

At the circuit level, the module Main 104 preferably comprises data-storage means 110 and data-processing means 112, provided by a first microprocessor, interfaced to which are the I/O devices and the bus control flags. An architecture of this sort is to be deemed within the reach of a person skilled in the sector. Its circuit implementation, once the necessary purposes and functions are known, does not present particular difficulties for a person skilled in the sector, and consequently it is not deemed necessary to provide any further technical details.

The user interface 114 is preferably constituted by an LCD 116 and a series of keys 118 for the functions commonly required. Optionally, it is possible to carry out remote control via a serial interface. It is to be understood that other types of interface are possible, for example a touch screen or the like.

The module Main 104 (namely, the data-storage means 110 and data-processing means 112) is moreover responsible for controlling synthesis of the data, said data comprising the aforementioned parameters Vi, and also second parameters T-packi, Freqi, T-sloti, that can be associated to each primitive waveform S0-18i, the meaning of which will be explained in detail. A further parameter of composition of the string, T-linki, can be associated to the geometries S16 to S18, as will be explained more fully hereinafter.

Advantageously, the set of primitive waveforms S00-S18, can be stored in a storage medium, whether internal and forming an integral part of the module Main 104 (non-volatile memory modules or the like) or else external and/or removable, such as for example a CD-ROM or the like.

A resident software processes continuously the sequence S to be digitalized, by sending on the bus 102 a set of data Bi, identifying said sequence S, necessary for the synthesizer module 106 to obtain, in real time, an electrical output signal Out, corresponding to the required sequence S. In particular, the software resident in the module Main 104 implements a selection algorithm, schematically illustrated in the diagram of FIG. 4A.

For purposes of clarity, FIG. 4B is a schematic illustration of the result of the selection algorithm. In the figure and in the sequel of the description the following definitions will be used:

Packet—Pack: succession of a single primitive waveform, repeated in time. The temporal duration T-packi of a packet Packi is of at least 700 ms, with an upper limit preferably of approximately 10 s. However, it is to be understood that the duration of a packet may even be longer than 10 s, in the limit equal to the duration of the treatment.

Intercycle pause—Slot: pause interval between one packet and the next, of a temporal duration T-sloti preferably ranging between 0 and 38 ms.

Link substring—T-link: it follows the pause and precedes the packet, and has a temporal duration T-linki preferably ranging between 0 and 235 ms.

Frequency—Freq: frequency to be associated to the waveform of the packet preferably ranging between approximately 43 and 52 Hz, said values corresponding to a period ranging between approximately 23.26 ms and approximately 19.23 ms.

The sequence S will hence be processed as composition of one or more of said primitive waveforms Si in temporal sequence, each of which is in turn processed on the basis of the parameters T-packi, Freqi, T-sloti, T-linki, which are calculated according to pre-set modalities that will be illustrated in the sequel of the description.

The geometry of each individual waveform Si graphically described in FIGS. 2A to 2S has an intrinsic information content such as to induce analgesia.

In this sense, performing a TENS of a traditional type that, instead of using the classic waveforms deriving from square waves, sinusoids, triangular waves, etc. supplies continuously even just one of the waveforms S00-S18 described herein would already constitute a considerable step ahead both from a technological standpoint and from the standpoint of the results.

Each further processing of the basic information of the individual waveforms described hereinafter, to form packets and then more complex strings of information, is, however, preferable for optimizing the analgesic effect in the most difficult cases, above all of chronic neuropathic and oncological pain, for which the device is explicitly designed, as well as in those cases that do not respond in a satisfactory way to any conventional pharmacological and/or electroanalgesic treatment, whether of a surface type or of an implanted type.

Before proceeding with the description, a short premise is, however, necessary. The C.N.S. by its very nature discriminates and processes information, but in this process also has the property of changing over time the perception of information into background noise if the content thereof is monotonic, i.e., always the same for long periods of time. An explicative analogy is what happens when we enter a crowded environment full of people who are talking. Initially, we tend to discriminate simultaneously one or more voices present in the surrounding environment, but over time the perceptive adaptation will lead us to consider the overall voices as environmental noise, i.e., background noise, ignoring the associated content of information, even though this is always present. Said situation changes only if the background noise changes abruptly, i.e., the monotony is broken by a new element that varies the average content of information, for example a person who suddenly raises his tone of voice, a plate that smashes on the floor, etc.

A similar problem applies to the use of traditional TENS and explains its known limits of effectiveness. Initially responsive patients, in time become resistant, and the therapy is no longer effective. Since the central role of control of the discriminating properties of the C.N.S. is always the information perceived, as in the case of the analgesic effectiveness in the various types of pain, it is necessary to synthesize different sequences of “non-pain” information, thus widening the dynamics of the string resulting therefrom and preventing monotony, in order for the treatment to be always effective. This principle has been verified experimentally in clinical practice with favourable outcome for the object and purposes of the present invention.

The strategy of the dynamic construction of information is elaborated by the module Main 104, which, by writing on the bus control bytes Bi, makes the information available to the synthesis module Synth 106, which, by reading the current byte generates accordingly the geometry required with the associated properties of frequency, intercycle pause, and duration of the packet.

Each control byte Bi contains at least the information on a single packet, namely:

a first portion of four bits for encoding the primitive waveform Si to be used for the current packet Packi;

a second portion of two bits for setting the frequency Freqi (43, 46, 49, 52 Hz) thereof; and

a third portion of two bits for setting the duration of the intercycle pause Tsloti (0-38 ms), subsequent to the current packet Packi, which controls also the selection of one of the geometries comprised between S16 and S18 used in T-linki.

The temporal duration T-packi of the packet is instead determined by the time in which the corresponding control byte Bi is kept unvaried and available on the bus.

The dynamic construction of the control byte Bi is carried out following probabilistic criteria, the reference parameters of which have been identified in the basic scientific research carried out by the author, which was preparatory to the development of the technology described. The processing core for the output probabilities in composing the control byte is a random generator interconnected to a probabilistic filter that modifies the output thereof in percentage terms. Basically, a pseudo-random number is initially generated continuously. This number passes through a conditional filter, which establishes the probability thresholds of the effective user. The code then carries out the necessary filtering in order for the arbitrary probability, which has been defined in order to modify the values of the variable P starting from a random generator, to be respected. This model is implicitly recalled in the ensuing descriptions, which regard the algorithm for construction of the control bytes, applying one or more of the conditional filters described hereinafter.

### Probability of Selection of the Primitive Waveform

The selection of the primitive waveforms Si is made on the basis of a first probabilistic criterion. Even though it is to be understood that said first criterion can in any case involve a completely random selection, according to the present invention, it is preferable to vary each time the probability of selection of each of the waveforms by dynamically varying a first probabilistic filter used for said purpose. This solution is mathematically necessary in order to reduce considerably the entropy of the information and hence render the clinical results of the treatment reproducible.

In particular, the first sixteen primitive waveforms are divided into four sets, each containing four different base waveform. Initially, assigned to each set is an equal output probability (25%) and assigned to each waveform associated to a set is an equal output probability (25%). When a set is selected, its output probability is reduced to 10%, that of the immediately subsequent set is automatically increased to 40%, and that of the remaining sets is brought to 25%, on a cyclic basis.

In practice, the selection of Set 1 implies setting its subsequent output probability at 10%, that of Set 2 at 40%, and that of Sets 3 and 4 at 25%. Likewise, the selection of Set 4 implies setting its subsequent output probability at 10%, that of Set 1 at 40%, that of the remaining sets at 25%, and so on.

The second step is modification of the probability of selection, within the set selected, of one of the four possible waveforms, which are initially equiprobable in one and the same set. The waveform selected, in association with the associated frequency, sends to 0% its subsequent output probability in the set, which is restored to 33.33% only when another waveform belonging to the same set and associated to the same frequency is selected, following the same procedure of modification of the subsequent output probability within the same set. In practice, before setting to zero its output probability in the absence of a general reset, each waveform has four different output possibilities in relation to the four possible associated frequencies. Consequently, even though it is a low-probability event, the consecutive output of one and the same waveform with different frequencies is possible, but the consecutive output of one and the same waveform with one and the same frequency is in no case possible.

It is to be noted that the association of the sixteen waveforms available to the four sets envisaged follows analytical criteria associated to experimental validation. One of the groupings that has proven experimentally most effective is the following:

Set 1: S00, S01, S02, S03

Set 2: S04, S05, S06, S07

Set 3: S08, S09, S10, S11

Set 4: S12, S13, S14, S15

However, for the purposes of the present invention, this must be considered valid in each of its possible combinations in so far as in any case the resulting rate of analgesic information is always present, albeit with different degrees of effectiveness.

As regards the further parameters indicated, T-packi, Freqi, T-sloti, T-linki, rules of selection of a probabilistic type are once again applied. In particular, the parameters are selected, starting from values, or ranges of values, pre-set on the basis of further and respective probabilistic criteria, which preferably are dynamically modified applying further probabilistic filters in order to vary each time the probability of selection of the pre-set starting values. This generates a behaviour of the system with high dynamic variability, but with precise trends that render it non-random.

Described hereinafter are the probabilistic filters corresponding to the aforementioned parameters, as preferably used in the present invention. Once again it is to be understood that said conditions can be modified, without thereby departing from the inventive idea underlying the present disclosure.

### Probability of Selection of the Frequency Associated to the Primitive Waveform Selected

It is envisaged that the four preferred frequencies to be assigned to the primitive waveform selected are the following:

43 Hz—15%,

46 Hz—45%,

30 49 Hz—15%,

52 Hz—25%.

As has been said above, the selection of one of the frequencies affects also the subsequent probability of selection of the waveforms, as described previously.

### Probability of Selection of the Intercycle Pause

The overall time of therapy is divided by 4, and distinguished into corresponding steps, in which the probability of selection of a duration is modified. The duration of the intercycle pause deriving therefrom in terms of probability is the following:

Step 1: 70%—0 ms, 30%—12 ms

Step 2: 70%—12 ms, 30%—25 ms

Step 3: 70%—25 ms, 30%—38 ms

Step 4: 70%—38 ms, 30%—0 ms

### Probability of Temporal Duration of a Packet

In this case, the randomization is simpler, and sets the temporal duration of a packet starting from a minimum of 0.7 s. The synthesizer module Synth 106 comprises in the first place means for generating an electrical output signal “Out”, corresponding to the sequence S as programmed by the module Main 104. The synthesis is made preferably for an 8-bit digital-to-analog conversion, once again controlled by a resident firmware.

With reference to the diagram of FIG. 5, there may be noted the use of two digital-to-analog converters (DACs) 120, 122 interconnected with a second microprocessor “mP Unit” 111 dedicated to the synthesis. The microprocessor 111 continuously reads on the bus the current control byte Bi generated and supplied by the module Main 104 and supplies, on the basis of the information contained therein, on the input port of the DAC identified in the figure as “DAC2” 122, the amplitude values (read by the corresponding vector S00-S18) to be converted for the synthesis of the waveform selected. Each individual sample is of course timed on the basis of the frequency Freqi selected.

The output of the DAC2 122, which is typically a stepwise output, is preferably integrated by a low-pass filter 123 constituted by an output operational amplifier, which functions also as buffer. In the embodiment described, the cut-off frequency of the filter 123 is calculated at approximately 1592 Hz, and its slope is of 6 dB/oct. At output, the signal Out is made available on the bus 102 for the channel modules 108 connected thereto. Preferably, the reference input of the converter DAC2 122 is not connected to a voltage source at a constant value as is customary, but is supplied by a second DAC, identified as “DAC1” 120.

The input port of the converter DAC1 120 is supplied with pre-programmed data in order to perform a rapid equalization of the response of the current-feedback circuits present on the subsequent channel module 108, including the precision rectifier, as a function of the various waveforms synthesized at that moment.

In the second place, in executing the code, the dynamic modification of the vectors resident in the non-volatile memory enables an amplitude modulation that performs different functions of control of effectiveness and patient compliance.

The amplitude modulation is mainly useful for increasing the noise figure, in terms of amplitude nonlinearity, present in long sequences of action potentials typical of the nerve cell subjected to prolonged stimuli. The overall dynamics of the output variation, considering 100% as the maximum amplitude, can indicatively drop to 67% of the upper limit.

As has already been said, the analog signal thus obtained is made available on the bus 102 for all the channel modules Chk 108 envisaged and connected thereto. A channel module Chk 108 can be obtained according to different architectures, from the one that envisages simply the use of a microprocessor to the one that envisages an extensive use of operational amplifiers and wired logics. This second choice, albeit requiring a greater number of components and involving greater circuit complexity, is preferred because it is intrinsically more stable and reliable, in addition to being less noisy from the processing standpoint and from the standpoint of the analog output. These requisites are fundamental for the safety of the patient undergoing treatment, and for this reason have priority over other industrial requirements.

The block diagram of FIGS. 6a-6b shows schematically the constitution of a generic channel module Chk 108 that has to carry out the required functions, which are substantially those of filtering, modulation, and amplification of the output signal supplied by the module Synth 106, feedback-regulation of the level of current of the output signal, necessary also for compensating variations of pressure on the electrodes 160 and effects of perspiration, and alarm in the case of detachment or short-circuiting of the external wiring present on the patient. The evolution of the complexity of the strings of information and of the corresponding modulations has rendered necessary the development of control circuits that are particularly innovative and unusual, hence readily recognizable as being specific of this invention. The block diagram of FIG. 6a explains in detail these functional details.

According to said block diagram, each channel module 108 comprises:
at least one buffer 124;
at least one digital amplitude control 126, connected to the buffer 124;
at least one band-pass filter 128, connected to the digital amplitude control 126;
at least one linear amplifier 130, connected to the band-pass filter 128;
at least one synchronism generator 132, connected to the digital amplitude control 126;
at least one comparator 134, connected to the digital amplitude control 126;
at least one activation-window control 136, connected to the digital amplitude control 126;
at least one low-pass filter 138, connected to the comparator 134 and to the activation-window control 136;
at least one sampling element 140, connected to the comparator 134;
at least one precision rectifier 142, connected to the sampling element 140;
at least one first separating transformer 144, connected to the precision rectifier 142;
at least one second separating transformer 146, connected to the linear amplifier 130; and
at least one protection and output-enabling element 148, connected to the first and second separating transformers 144, 146.

With reference to the diagram of FIG. 6a, explained in detail hereinafter are the corresponding circuit functions.

The signal generated by the module Synth 106 is collected at input and buffered (in 124), so that the parallelism of a number of channels does not generate coupling problems. The digital amplitude control performs three important functions: 1) automatic regulation of the output as the load varies on the value set manually by the purposely designed potentiometric level control; 2) contribution to the amplitude sub-modulations, which would not otherwise be managed digitally directly by the module Synth 106; 3) safety reactions with reset of the output signal in the event of detection of functional faults.

It is to be noted that a quartz synchronism generator 132 (common to all the channels) controls the variation of the digital amplitude control 126, supplying a very precise temporal reference Vref of the activity of said control. This temporal reference Vref enables passage of the amplitude sub-modulations without interfering with the process of current regulation that compensates for any dynamic imbalance of the load or of the variations of the waveforms. A similar temporal reference synchronized to the previous one, generated once again in the synchronization block 132, modulates periodically and slightly in amplitude (<10%) the reference set manually of the intensity of the output stimulus, thus obtaining one of the necessary sub-modulations not generated digitally in the module Synth 106.

For the same reasons of harmonizing two requirements that are deeply in contrast at a technical level, i.e., that of regulating precisely the output current but at the same time not preventing passage of the modulations necessary for effectiveness of the invention, also other parts of this circuit operate in a way different from the standards normally adopted and specific for these activities. As regards the comparator 134, it may be noted that the determination of the direction in which the amplitude of the signal is modified (increment/decrement) is made instantaneously in response to the feedback loop, which, together with the level set manually, constitutes the other input point of the comparator 134. This change of direction of the regulation is prepared, but is not active until the consensus of the control of activation of the regulation is received. The control of activation analyzes the oscillations of the comparator 134 and discriminates the useful part thereof, first through the low-pass filter 138, and then with the window discriminator 136. When the signal resulting from this processing path identifies the effective need of regulation of the deviation of the current measured with respect to the fixed one, the consensus to the modification of the amplitude of the signal that is useful for compensating the difference found is generated. When, instead, the process of analysis identifies a sub-modulation that must be passed, the consensus to the variation is denied. Finally, the digital amplitude control 126 can be forced by software to a temporary modulation downwards or to reset of the output signal using the control bit “Set Zero”.

The signal thus processed is sent to the band-pass filter 128, which has the purpose of eliminating spurious modulations, and completing the geometrical definition of the waveforms at output, as described more fully visually in the attached figures. A linear power amplifier 130 drives the second separating output transformer 146, and through a further patient-protection 148 system (varistors, limiting resistors, and relays), finally supplies the signal with the characteristics suitable for clinical use on the patient.

From the output, through the first separating transformer 144, a small portion of the signal is taken across the shunt resistor 143, said portion of signal being necessary for evaluation and regulation of the effective current supplied to the patient. This portion of signal must be converted into a d.c. voltage that can be used for the purpose, but also in this case it is not possible to use conventional circuits for reasons of functional synergy with the modules described previously.

The precision rectifier 142 that receives the signal duly insulated by the galvanic-separation transformer 144, admits of two possible solutions. The first, the simpler one, provides a filtering based upon just one integration system, which is calculated so as to allow passage of the modulations albeit maintaining control of the average current supplied within the limits fixed manually. The single time constant, synergized with the other parameters of sampling synchronism, is exploited also for a particular amplitude modulation that occurs at the start of each change of sequence for a very short time (<300 ms). The limit of this system is that for given changes of sequence the settling time can produce sensations that are at times somewhat abrupt, even though they are altogether harmless and always comprised within the stimulation parameters envisaged.

A variant of this circuitry, visible in the block diagram of FIG. 6b, enables minimization of this discomfort for the patient, operating with two different time constants, one for the negative half-wave and one for the positive half-wave, which are integrated in a differential circuit before being sampled.

According to this block diagram, the precision rectifier 142 is constituted by:
at least one (+) peak detector 150-1;
at least one (+) integrator 152-1, connected to the (+) peak detector 150-1;
at least one (−) peak detector 150-2;
at least one (−) integrator 152-2, connected to the (−) peak detector 150-2;
at least one differential amplifier 154, connected to the (+) and (−) peak detectors 150-1, 150-2; and

at least one buffer amplifier 156, connected to the differential amplifier 154.

This original circuitry, unlike the previous one, introduces a very specific nonlinear behaviour, which harmonizes perfectly with the requirements of effectiveness and compliance of the device for which this updating has been required. In brief, this particular precision rectifier, together with the link sequence T-linki, introduces at the start of each new packet an equal amplitude modulation that is effective and readily recognizable by the patient, but perceived as “softer”, hence not alarming or likely to generate discomfort. At the same time, this circuit is particularly efficient in maintaining the average current values set manually over a wide range of load impedances, without causing any alteration of the modulations and of the geometries used for constructing the synthetic non-pain information, eliminating the discomfort that the patient could perceive in the regulation step, due to the temporary spurious stimulation of the A-Delta fibres. This characteristic of considerable stability of the feedback in very different operating and modulation conditions has also rendered the device substantially independent of the various types of electrodes used.

To understand more fully the importance of a wide dynamic range of regulation of these circuits given a variable load, it should be borne in mind that the FDA evaluation standard, which is the most severe currently in use, requires an evaluation of the maximum current that can be supplied by the device on a standard load of 500Ω. This evaluation implies the use of electrodes for TENS, which exhibit a very low resistance, and hence substantially falling within the fictitious load of 500Ω necessary for this evaluation, which represents the average impedance of the human body undergoing electrostimulation. The device according to the present invention cannot use TENS electrodes because they are excessively wide, in so far as the TENS must stimulate the nerve, whereas the device according to the present invention has as target small dermatometric areas where the surface receptors of the C fibres are recruited, and not the nerve. For this reason, right from the outset it has been decided to use ECG electrodes, which in terms of dimensions of the surface of electrical contact, practicality of use, and hygiene (they are disposable) are optimally suited to the purposes envisaged. The problem to be solved was the different characteristic of electrical impedance, further modified during use by the passage of currents not envisaged in the original use. To the present day, for a correct operation of the regulation system, it has been necessary to dimension its exact behaviour in an impedance range that may vary dynamically from 100 to 10 000Ω, so as not to be affected by the different constructional characteristics of the electrodes, and their variability of behaviour during stimulation. FIG. 8 illustrates an example of these electrodes 160.

Whatever the solution adopted for the precision rectifier, the final processing of the feedback signal is performed always through a programmed sampling. The sampling carries out stabilization of the response of the circuit, via further synchronization with the real-time synthesis of the waveforms currently generated by the module Synth 106.

The channel module 108 can be replicated so as to extend the number of outputs available for the user. It is consequently possible to envisage the use of one or more channel modules 108 (preferably five or more), all exactly the same as one another and controlled as described above.

By “string of non-pain information” is hence understood the temporal sequence of the packets and pauses, with the characteristics of modulation more fully described above, of which FIG. 7 constitutes an example.

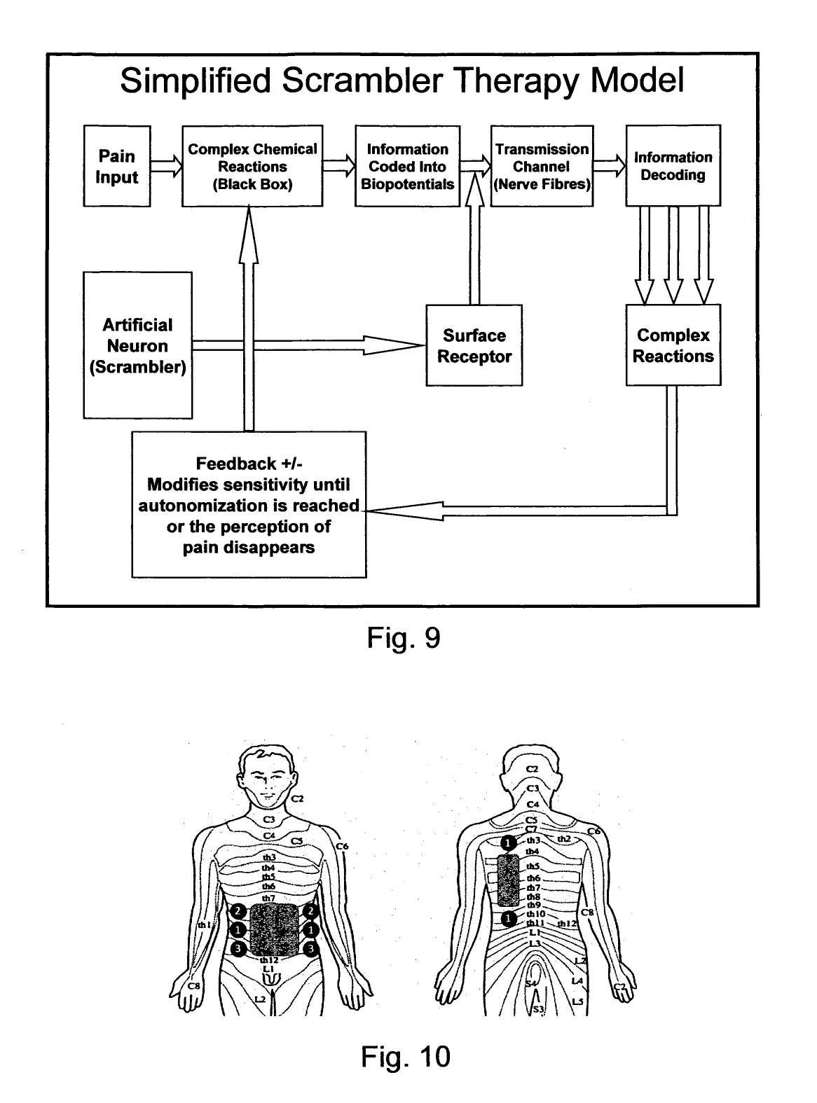

FIG. 9 is a schematic illustration of the clinical functioning of the inventive apparatus.

The method of operation of said apparatus comprises the following steps:
pain input
complex chemical reactions (black box)
information encoded into biopotentials
transmission channel (nerve fibres)
information decoding
complex reactions
feedback: modification of the sensitivity until autonomization is reached or the perception of pain disappears.

Via appropriate digital warnings coming from the channel modules 108, the module Main 104 can moreover verify proper operation and the possible presence of critical failure, automatically interrupting the treatment.

The safety of the patient is guaranteed by three simultaneous levels of circuit responses in the case of faults or operative errors, as well as in the event of failure. The first protection level is of a software type, obtained through monitoring of purposely provided warnings read by the module Main 104. The second protection level is internal to the channel module 108, and is based upon responses, which are obtained directly from wired logic and are hence not sensitive to any possible blockage of execution of the program. The third protection level is of a passive type, and guarantees, even in the event of serious failures, that limit currents for the patient are not exceeded, thanks to the output resistive network, a branch of which can be varied via varistors, and to the precise sizing of the coupling transformers.

From a therapeutic standpoint, the present invention is indicated in all cases of severe pain, chronic pain, drug-resistant pain, pain resistant to opiates, TENS, or implanted stimulators, whether of a benign type of or an oncological type, and can moreover be applied in the paediatric sector.

In the conditions of proper use recommended, which will be described more fully in what follows, the analgesia is extremely fast. Only a few seconds after start of the treatment are sufficient for completing the step of regulation of the intensity of the stimulus and obtaining complete disappearance of the perception of pain, even pain of extreme intensity and not responsive to opiates. The prolonged use increases the effectiveness of the treatment, with progressive rise of the pain threshold beyond the same and increase of the duration in hours of the analgesic effect. In the scientific literature, this characteristic is unique in so far as a further limit of conventional electro-analgesia is the development of habituation to the treatment, with progressive loss of effectiveness over time. During the clinical experiments, no undesirable effects were encountered in the conditions of use recommended.

In order to implement the therapeutic analgesic methodology, the apparatus 100 according to the present invention can be used in the hospital and in the surgery, as well as in out-patient and free-living conditions, even under the patient's own management, obviously always with the consultancy of a physician. The optimal duration of treatment that guarantees, in addition to immediate effectiveness, a prolonged duration of analgesia is 30-45 minutes.

In the case of oncological pain in the terminal phase, except for particular reasons, the treatment in the patient would should be conducted according to the needs.

When the treatment is carried out, it is expedient to reduce gradually, as far as possible, the analgesic support of a pharmacological type. It has been experimentally found that it is possible to suspend completely analgesic drugs in the majority of cases of very severe or untreatable oncological pain, whereas, in the remaining cases, it is possible to reduce considerably the dosage of opium-based substances, or to replace them with other less invasive drugs. This precaution is necessary not only for optimizing the effects of the treatment, but also for improving the quality of life of the patient, which is the main purpose of palliative treatment.

In the case of benign pain, the treatment should envisage (possibly repeatable) cycles consisting of ten treatments at a rate of five per week.

A particular case is that of patients who use anticonvulsants for analgesic purposes. In this case, the responses are normally slower and less stable over time. It is likely that the reduction of effectiveness is due to the depression of the cerebral bioelectrical activity induced by the anticonvulsant, which antagonizes the active ingredient of the procedure. The gradual reduction of the anticonvulsants can determine rebound effects, especially if it is excessively fast. Recently, there has also been noted an unfavourable interaction in terms of effectiveness with ketamine used for analgesic purposes. This unfavourable association is concordant with the active ingredient used in so far as ketamine is not an analgesic drug, but a powerful anaesthetic.

Optimal adjuvant drugs, if necessary, generally belong to the category of FANS or Paracetamol. The use of opiates does not reduce the effectiveness in the course of treatment, but if not eliminated during the therapeutic cycle, can prevent a favourable remodulation of the pain threshold upwards, and produce responses less stable over time at the end of the cycle.

The therapy forming the subject of the present invention is an extraordinarily effective system for controlling pain provided that it is used correctly, following the rules illustrated below. Experimentally, it has been noted that in almost all cases in which a satisfactory response to the treatment was absent, this was due exclusively to the erroneous dermatometric arrangement of the electrodes 160, or to non-perfect positioning thereof. Once the errors were removed, the effectiveness returned to being the one expected.

For a good operativeness it is preferable to use disposable 5-cm electrodes 160 of the ECG type or ones having an equivalent surface. Excessively small electrodes 160 may cause irritation, whereas excessively large ones can recruit more nerve terminations than what is necessary. If the surface to be treated is extensive, it is possible to use a number of channels. Each disposable electrode, even though it is already pre-treated, must preferably be coated with conductive gel on the surface of electrical contact with the patient.

The parts of the body where the electrodes 160 are to be positioned must not be cleaned with alcohol or other dehydrating substances, and must be perfectly dry to enable proper adhesion of the electrode 160. A poor contact, in addition to rendering the treatment less effective, may cause troublesome irritation.

Finally, it is necessary not to position the electrodes 160 on irritated or sore areas or on biological liquids and, as a general rule, to connect the wires on the electrodes 160 only after they have been positioned properly.

Except for particular neurological damage, the electrodes 160 are to be arranged immediately at the sides of the painful area, following for their arrangement the prevalent geometry of the pain (horizontal, vertical, diagonal).

The electrodes 160, except in particular cases, should never be positioned inside the painful area. This precaution depends upon the fact that presumably the receptors involved in the painful area can exhibit morpho-functional anomalies produced by the neuropathic damage. A general event can prevent proper transmission of the non-pain information, preventing the expected analgesic response. FIG. 10 shows two examples of positioning of a pair of electrodes 160.

Imagining a straight line that passes between the two points represented by the electrodes 160, said points must pass indicatively at the centre of the area of maximum pain. Where necessary, it is advisable to use a number of channels to cover very extensive painful areas, respecting the electrical phases, which can be identified for example through a conventional polarity, distinguished for example by a different colour of the electrodes 160 (e.g. red and black) or else in some other way (+/−, etc.). Hence, in general, it is necessary to position all the electrodes 160 of one and the same type (same colour, etc.) on one and the same side. For simplicity, in the figures, the electrodes 160 are conventionally identified with the symbols “+” and “−”.

Consequently, if a number of channels are used, all the vertical positionings must have at the top and at the bottom the electrodes 160 of the same type of each channel. The same applies to the horizontal or diagonal positionings, which for each channel must have both on the right and on the left electrodes 160 of the same type; otherwise, there is loss of effectiveness.

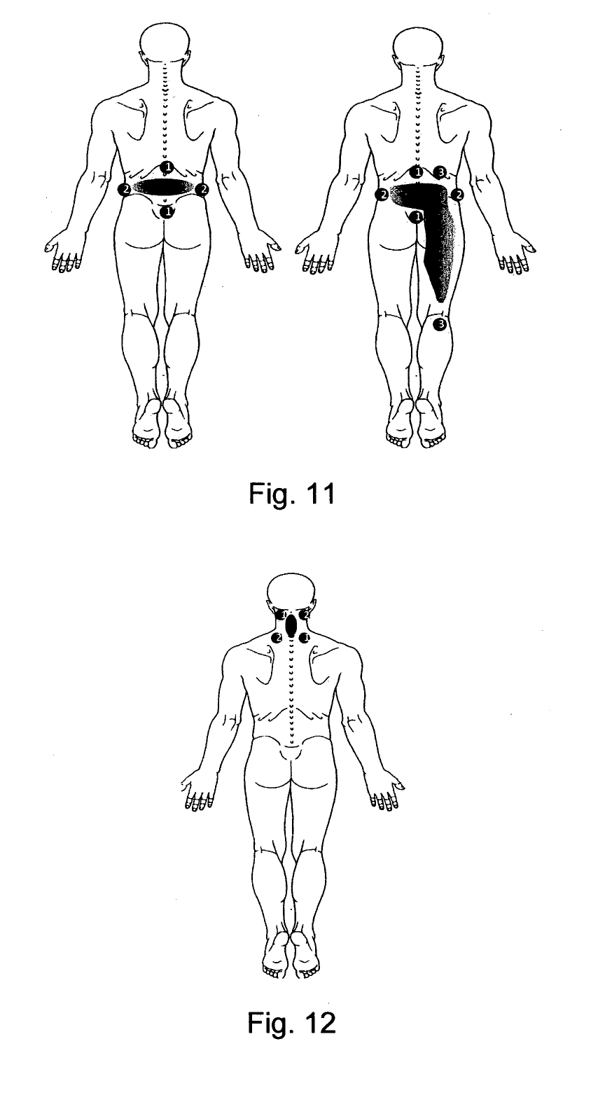

It is moreover possible, following the same rules, to perform mixed positionings, horizontally and vertically, as illustrated in the representations of FIG. 11.

At times, in fact, it may prove problematical to find the right arrangement of the electrodes 160 on account of modifications of the innervation due to neuropathic conditions, traumas, or surgical interventions, or other modifications of the pain system induced by chronicization. In this case, it is necessary to proceed by redundancy and by trial and error, bearing in mind that it is possible to understand immediately when the positioning is correct because the pain disappears immediately in the area treated properly. Using this type of feedback it is possible to solve even the most complex situations thanks to the immediate effect on the pain symptom.

In some cases, it is possible to encounter difficulties in identifying areas free from pain that are useful for treatment. In these circumstances, it is possible to adopt advanced positioning strategies, which normally solve the problem. A first strategy, which is especially useful in facial pain, is that of using the contralateral ways. In the case of lack of response, usually it is possible to adopt a homolateral positioning for one of the two electrodes 160 of the channel 108 used, and a contralateral positioning for the other electrode of the pair.

Another type of positioning that frequently solves difficult situations in a very simple way is of a crossed type, in combination, when necessary, with the traditional vertical/horizontal/diagonal positioning, using the other free channels.

The latter type of positioning is represented in the illustrations of FIG. 12. Of course, it is to understood that, aside from the illustrations provided by way of example, all the types of positioning described can be validly applied to any area of the body.

It should be recalled that the index of a correct treatment is only the complete and immediate disappearance of pain in the treated areas. For this reason it is not possible to treat pain before its appearance.

In addition, in the case of pains that appear only in given positions, its is necessary to make sure that the positioning and the verification of its effectiveness are made always in the conditions in which the pain is present; otherwise the therapy can certainly not be considered effective. Once the correct positioning is certain, the patient can assume the positions that he prefers in order to proceed with the treatment.

It is moreover necessary to test always one channel at a time in succession, positioning one pair of electrodes 160 at a time, ensuring their analgesic effectiveness, and proceeding in the same way to eliminate any possible residual pain in the areas not covered by the previous pair. Pairs of electrodes 160 that are not effective are to be eliminated and repositioned to obtain the expected analgesia. If the remaining treatment time has been reduced excessively, it is sufficient to set to zero the levels of each channel without modifying the positioning of the electrodes, then interrupt the treatment with the appropriate commands, and finally start it off again to carry out a complete treatment properly.

In the majority of applications, beneficial and positive effects are encountered already after a very short treatment. However, it is preferable for the treatment to last at least 30 min. In the cases of very intense pain, typically oncological pain, the optimal value should preferably be brought up to 45 min. The treatment starts automatically when the level of any of the channels rises, and stops autonomously when the pre-set time elapses; said time can be modified in the setup step, if necessary.

The treatment is completely automated and does not require any individual setting of wave parameters, such as for example frequency, duty cycle, scanning, etc., also because they are not significant in the active ingredient used. The only manual regulation necessary is the regulation of the amplitude of the stimulus to adapt it to the individual sensitivity of the patient and to the correct perception of sensations that identify clearly the recruitment of the C fibres, together with the disappearance of pain in the area covered.

For said purpose, the amplitudes of the channels must preferably be regulated at the limit of the individual threshold of tolerability that the patient treated subjectively perceives.

The levels must be initially regulated during the first moments of treatment, and adjusted whenever the stimulus is no longer perceived with the same intensity on both the electrodes 160 of each channel 108 involved as a result of the progressive analgesia.

In the muscular pain, even though this is a secondary target for this type of therapy specifically studied for neuropathic and oncological pain, for a better effectiveness, at times it may be preferable that, in addition to what has been expressed as regards the recommendations on positioning (in general sufficient), the flow of current is perceived between the pairs of electrodes 160 of one and the same channel 108 when correct positioning is certain. If, notwithstanding this, the response were not to be good, it is then preferable to use more than one channel for the same area.

To prevent effects of “rebound” during or after therapy it is necessary always to make sure that the patient does not perceive, at one or more electrodes 160, a painful sensation and/or an extremely unpleasant sensation, which is an expression of a residual recruitment of fibres still in connection with the painful area. Normally, this occurs when the simultaneous use of analgesic drugs can mask the effective area of pain. This sensation is easy to recognize because the synthesis of the “non-pain” information (the desired one) is in general optimally tolerated, and the sensation associated thereto is frequently defined as pleasant. In this case, the electrodes 160 are to be repositioned slightly further away from the point chosen until the problem is eliminated and effective analgesia obtained. The failure to respect this recommendation may give rise to undesirable rebound effects, during or after treatment.

A further and important check in order to know whether the positioning is correct from an electrical and functional standpoint, is that of asking the patient being treated, after activation of each channel, whether in that particular sector the sensation of pain varies. In fact, irrespective of the initial intensity of the pain, which may even be extremely high, the answer should always be negative (no perception of pain=optimal positioning) immediately after proper regulation of the intensity of the stimulus.

If, upon complete activation of all the channels, the patient still reports pain, even if attenuated, the covering is not complete and the therapy will produce effects that are significantly inferior to what is necessary and possible to achieve. Incomplete analgesia depends upon the other than perfect centring of the innervations involved, or upon the fact that the area is very extensive area and not completely treated. In the former case, the electrodes 160 should be positioned better, as described more fully in the various positioning strategies. In the latter case, it is necessary to use other channels, as explained above.

If it is not possible to modify the positioning, and after a few minutes of treatment, preferably after approximately five minutes, there still remains the perception of pain, albeit attenuated, the result will not be good. In the course of treatment there should always be immediate elimination of the perception of pain, even if this is of extremely high degree.

If, at the end of the treatment, the pain, albeit not present during application, re-appears (even in an attenuated form) or recidivates after few minutes, it is necessary to repeat the application ensuring that all the steps described above are respected.

The present invention has been described so far with reference to a preferred embodiment. It is to be understood that other embodiments may exist that draw upon the same inventive idea, all of which fall within the sphere of protection of the claims appearing hereinafter.

1. An apparatus for rapid pain suppression, comprising:
a main module, comprising data-storage means and data-processing means;
a synthesizer module; and
one or more channel modules;
wherein:
the data-storage means contain data comprising:
first parameters, which identify a set of primitive waveforms, each primitive waveform having a periodic and predetermined time plot;
second parameters, which can be associated to each of the primitive waveforms;
the data-processing means are designed and configured so as to process a set of data which identify a sequence made up of one or more of the primitive waveforms in temporal sequence, each of the primitive waveforms of the sequence being processed on the basis of one or more of the second parameters;
the synthesizer module comprises means for generating an electrical output signal corresponding to the sequence; and
the one or more channel modules comprise means for application of the electrical output signal to a body using C fibers as primary vehicle for inducing analgesia, without blocking a conduction thereof of the C fibers, so as to excite the C fibers in order to convert the electrical stimulus into non-pain information in the C fibers themselves.

2. The apparatus of claim 1, wherein the first parameters comprise values of amplitude of each primitive waveform of the set of primitive waveforms.

3. The apparatus of claim 2, wherein each of the primitive waveforms is represented in digital format by a corresponding vector of values, expressed in the hexadecimal system:
V0=B6 FE FE FE FE FE FE FE FE FE FE FE FE FE FE FE EC BY C8 B6 A4 92 7F 00 20 40 60 6E 80 80 80 80 80 80 80 80 80 80 80 80 80 80 80 80 80 80 80 80 80 80 80 80 80 80 80 80 80 80 80 80 80 80 80 80
V1=81 B6 FE FE FE FE FE FE FE FE FE FE FE FE FE FE FE FE FE FE FE FE FA EC DE D0 C2 B4 A6 9A 8E 00 20 40 60 6E 80 80 80 80 80 80 80 80 80 80 80 80 80 80 80 80 80 80 80 80 80 80 80 80 80 80 80 80
V2=81 AA D4 FE FE FE FE FE FE FE FE FE FE FE FE FE FE FE FE FE FE FE FE FE FE FE F0 E2 D4 C6 B8 AA 9C 8E 80 00 20 40 60 6E 80 80 80 80 80 80 80 80 80 80 80 80 80 80 80 80 80 80 80 80 80 80 80 80
V3=81 AA D4 FE FE FE FE FE FE FE FE FE FE FE FE FE FE FE FE FE FE FE FE FE FE FE FE FE F5 EC E3 BY D1 C8 BF B6 AD A5 9B 92 80 00 20 40 60 6E 80 80 80 80 80 80 80 80 80 80 80 80 80 80 80 80 80 80
V4=B6 FE FE FE FE FE FE FE FE FE FE FE FE FE FE FE EC BY C8 B6 A4 92 80 00 10 20 30 40 60 70 80 80 80 80 80 80 80 80 80 80 80 80 80 80 80 80 80 80 80 80 80 80 80 80 80 80 80 80 80 80 80 80 80 80
V5=81 B6 FE FE FE FE FE FE FE FE FE FE FE FE FE FE FE FE FE FE FE FE FA EC DE D0 C2 B4 A6 9A 8E 00 10 20 30 40 60 70 80 80 80 80 80 80 80 80 80 80 80 80 80 80 80 80 80 80 80 80 80 80 80 80 80 80
V6=81 AA D4 FE FE FE FE FE FE FE FE FE FE FE FE FE FE FE FE FE FE FE FE FE FE FE F0 E2 D4 C6 B8 AA 9C 8E 80 00 10 20 30 40 60 70 80 80 80 80 80 80 80 80 80 80 80 80 80 80 80 80 80 80 80 80 80 80
V7=81 AA D4 FE FE FE FE FE FE FE FE FE FE FE FE FE FE FE FE FE FE FE FE FE FE FE FE FE F5 EC E3 BY D1 C8 BF B6 AD A5 9B 92 80 00 10 20 30 40 60 70 80 80 80 80 80 80 80 80 80 80 80 80 80 80 80 80
V8=B6 FE FE FE FE FE FE FE FE FE FE FE FE FE FE FE EC BY C8 B6 A4 92 80 00 04 08 0C 10 16 1C 22 28 2E 34 3A 40 50 60 70 78 80 80 80 80 80 80 80 80 80 80 80 80 80 80 80 80 80 80 80 80 80 80 80 80
V9=81 B6 FE FE FE FE FE FE FE FE FE FE FE FE FE FE FE FE FE FE FE FE FA EC DE D0 C2 B4 A6 9A 8E 00 04 08 0C 10 16 1C 22 28 2E 34 3A 40 50 60 70 78 80 80 80 80 80 80 80 80 80 80 80 80 80 80 80 80
V10=81 AA D4 FE FE FE FE FE FE FE FE FE FE FE FE FE FE FE FE FE FE FE FE FE FE FE F0 E2 D4 C6 B8 AA 9C 8E 80 00 04 08 0C 10 16 1C 22 28 2E 34 3A 40 50 60 70 78 80 80 80 80 80 80 80 80 80 80 80 80
V11=81 AA D4 FE FE FE FE FE FE FE FE FE FE FE FE FE FE FE FE FE FE FE FE FE FE FE FE FE F5 EC E3 BY D1 C8 BF B6 AD A5 9B 92 80 00 04 08 0C 10 16 1C 22 28 2E 34 3A 40 50 60 70 78 80 80 80 80 80 80
V12=B6 FE FE FE FE FE FE FE FE FE FE FE FE FE FE FE EC BY C8 B6 A4 92 89 00 05 09 0E 18 1E 20 22 28 2E 34 3A 40 49 52 5B 64 6D 77 7F 80 80 80 80 80 80 80 80 80 80 80 80 80 80 80 80 80 80 80 80 80
V13=81 B6 FE FE FE FE FE FE FE FE FE FE FE FE FE FE FE FE FE FE FE FE FA EC DE D0 C2 B4 A6 9A 8E 00 05 09 0E 18 1E 20 22 28 2E 34 3A 40 49 52 5B 64 6D 77 7F 80 80 80 80 80 80 80 80 80 80 80 80 80
V14=81 AA D4 FE FE FE FE FE FE FE FE FE FE FE FE FE FE FE FE FE FE FE FE FE FE FE F0 E2 D4 C6 B8 AA 9C 8E 80 00 05 09 0E 18 1E 20 22 28 2E 34 3A 40 49 52 5B 64 6D 77 7F 80 80 80 80 80 80 80 80 80
V15=81 AA D4 FE FE FE FE FE FE FE FE FE FE FE FE FE FE FE FE FE FE FE FE FE FE FE FE FE F5 EC E3 BY D1 C8 BF B6 AD A5 9B 92 80 00 05 09 0E 18 1E 20 22 28 2E 34 3A 40 49 52 5B 64 6D 77 7F 80 80 80
V16=B6 FE FE FE FE FE FE FE FE FE FE FE FE FE FE FE EC BY C8 B6 A4 92 81 00 11 23 34 3F 52 59 61 63 65 67 69 6B 70 75 7B 7D 80 80 80 80 80 80 80 80 80 80 80 80 80 80 80 80 80 80 80 80 80 80 80 80
V17=81 B6 FE FE FE FE FE FE FE FE FE FE FE FE FE FE FE FE FE FE FE FE FA EC DE D0 C2 B4 A6 9A 8E 00 11 23 34 3F 52 59 61 63 65 67 69 6B 70 75 7B 7D 80 80 80 80 80 80 80 80 80 80 80 80 80 80 80 80
V18=60 AA D4 FE FE FE FE FE FE FE FE FE FE FE FE FE FE FE FE FE FE FE FE FE FE FE F0 E2 D4 C6 B8 AA 9C 8E 81 00 11 23 34 3F 52 59 61 63 65 67 69 6B 70 75 7B 7D 80 80 80 80 80 80 80 80 80 80 80 80.

4. The apparatus of claim 1, wherein the data-storage means and data-processing means are designed to generate the set of data, in digital format, each datum comprising at least:
a first portion, identifying the primitive waveform selected to be repeated in succession in the sequence, to form a packet;
a second portion, identifying a frequency value to be associated to the primitive waveform selected in the sequence; and
a third portion, identifying a first value of temporal duration to be associated to an interval of pause, subsequent to the packet.

5. The apparatus of claim 4, wherein the data-storage means and data-processing means are designed to calculate moreover a second value of temporal duration to be associated to the duration of the packet.

6. The apparatus of claim 1, wherein the data-storage means and data-processing means are designed to select the waveforms with which to compose the sequence has to be composed, on the basis of a first criterion of a probabilistic type.

7. The apparatus of claim 6, wherein the first probabilistic criterion involves the variable and dynamic selection of the waveforms.

8. The apparatus of claim 6, wherein the first probabilistic criterion is dynamically modified according to a first probabilistic filter based upon pre-set rules in such a way as to vary in a foreseeable and organized manner the probability of selection of each of the waveforms.

9. The apparatus of claim 1, wherein the data-storage means and data-processing means are designed to calculate the second parameters for each waveform included in the sequence, on the basis of further probabilistic criteria, starting from pre-set values.

10. The apparatus of claim 9, wherein the further probabilistic criteria for calculating the second parameters are dynamically modified according to respective further probabilistic filters based upon corresponding pre-set rules in such a way as to vary the probability of selection of the pre-set values.

11. The apparatus of claim 1, wherein the main module processes each datum of the set of data in digital format, making it available as input for the synthesizer module.

12. The apparatus of claim 11, wherein each datum of the set of data in digital format is represented by a byte.

13. The apparatus of claim 1, wherein the data-storage means and data-processing means, comprised in the main module, include a first programmable microprocessor, designed to process data on the basis of firmware stored in corresponding storage devices.

14. The apparatus of claim 1, wherein the means for generating an electrical output signal, comprised in the synthesizer module, include a second microprocessor, designed to read the data supplied by the main module, and a first digital-to-analog converter, designed to convert the data received at input from the second microprocessor into an analog signal corresponding to the sequence.

15. The apparatus of claim 14, wherein the means for generating an electrical output signal further comprise a second digital-to-analog converter, designed to produce a modulating signal based upon pre-programmed data supplied by the second microprocessor and used as reference for the first digital-to-analog converter, thus carrying out an amplitude modulation of the electrical output signal.

16. The apparatus of claim 1, wherein the means for application of the electrical output signal, comprised in each of the channel modules, comprise:
a stage for filtering and amplification of the electrical signal issued by the synthesizer module;
a stage for feedback regulation of the level of current of the output signal;
a safety electrical-decoupling stage; and
devices for application of the regulated output signal.

17. The apparatus of claim 16, wherein each of the channel modules further comprises a stage for amplitude modulation of the electrical output signal.

18. The apparatus of claim 17, wherein the modulation is activated cyclically on just one of the one or more channel modules.

19. The apparatus of claim 1, further comprising a common bus for connection of, and exchange of data between, the main module, the synthesizer module, and the one or more channel modules.

20. The apparatus of claim 1, wherein the one or more channel modules are designed to perform the functions of filtering, modulation, and amplification of the output signal issued by the synthesizer module, feedback-regulation of the level of current of the output signal, necessary also for compensating variations of pressure on electrodes, effects of perspiration, alarm in the case of detachment or short-circuiting of the external wiring.

21. The apparatus of claim 1, further comprising at least one quartz-type synchronism generator, common to all the channels, designed to control the swing of a digital amplitude control, supplying a very precise temporal reference of the activity of the digital amplitude control.

22. The apparatus of claim 21, wherein each channel module comprises:
at least one buffer;
the digital amplitude control, connected to the buffer;
at least one band-pass filter, connected to the digital amplitude control;
at least one linear amplifier, connected to the band-pass filter;
the synchronism generator, connected to the digital amplitude control;
at least one comparator, connected to the digital amplitude control;
at least one activation-window control, connected to the digital amplitude control;
at least one low-pass filter, connected to the comparator and to the activation-window control;
at least one sampling element, connected to the comparator;
at least one precision rectifier, connected to the sampling element;
at least one first separating transformer, connected to the precision rectifier;
at least one second separating transformer, connected to the linear amplifier; and
at least one protection and output-enabling element, connected to the first and second separating transformers.

23. The apparatus of claim 22, wherein the precision rectifier is comprises:
at least one (+) peak detector;
at least one (+) integrator, connected to the at least one (+) peak detector
at least one (+) peak detector;
at least one (−) integrator, connected to the at least one (−) peak detector;
at least one differential amplifier, connected to the (+) and (−) peak detectors; and
at least one buffer amplifier, connected to the differential amplifier.

24. A method for activating the apparatus of claim 22, comprising the steps of:
pain input
complex chemical reactions
information encoded into biopotentials
transmission channel via nerve fibers
information decoding
complex reactions
feedback modification of the sensitivity until autonomization is reached or the perception of pain disappears.

25. The method of claim 24, wherein the signal generated by the synthesizer module is collected at input and buffered so that a parallelism of a number of channels does not generate problems of coupling, the digital amplitude control performing three functions: 1) automatic regulation of the output as a load on the value set manually by the purposely designed potentiometric level control varies; 2) contribution to an amplitude sub-modulations not otherwise managed digitally directly by the synthesizer module; 3) safety reactions with resetting of the output signal in case of detection of functional anomalies.

26. The method of claim 24, wherein a temporal reference issued by the quartz synchronism generator enables passage of amplitude sub-modulations without interfering with a process of regulation of current that compensates dynamic imbalances of a load or of variations of waveforms, the temporal reference, which is synchronized to the previous one, generated once again in the synchronization block, modulating periodically and slightly in amplitude the reference set manually of an intensity of the stimulus at output, thus obtaining one of the necessary sub-modulations that are not generated digitally in the synthesizer module.

27. The method of claim 24, wherein a determination of the direction in which to modify an amplitude of the signal as increment/decrement is made instantaneously in response to the feedback loop, which, together with a level set manually, constitutes another input point of the comparator, this change of direction of the regulation being prepared, but not being activated until a consensus of the control of activation of the regulation is received, the control of activation analysing oscillations of the comparator and discriminating a useful part of the oscillations, first through the low-pass filter, and subsequently with the window discriminator, wherein, when the signal resulting from this processing path identifies the effective need to regulate a deviation of the current measured with respect to a fixed current, a consensus for modification of the amplitude of the signal that is useful for compensating a found difference is generated, whereas, when the process of analysis identifies a sub-modulation that must be made pass, a consensus for the variation is denied.

28. The method of claim 24, wherein the signal processed is sent to the band-pass filter, which is designed to eliminate spurious modulations and complete a geometrical definition of the waveforms at output, whilst the linear power amplifier drives the second separating output transformer and, through a further system of protection, made up of varistors, limiting resistors, and relays, supplies the signal with characteristics suitable for use.

29. The method of claim 24, wherein, from the output through the first separating transformer, a small portion of the signal is taken across a shunt resistor, a portion of signal being necessary for evaluating and regulating an effective current supplied and being converted into a d.c. voltage that can be used for such purpose.

30. The method of claim 29, wherein the precision rectifier receives the signal duly insulated from the first galvanic-separation transformer and carries out a filtering based upon just one integration system, which is calculated so as to allow the modulations to pass albeit maintaining the control of an average current supplied in manually-fixed limits, a single time constant, synergized with other parameters of synchronism of sampling, being exploited also for a particular amplitude modulation that is made at a start of each change of sequence for a short time of less than 300 ms.

31. The method of claim 29, wherein two different time constants are used, one for the negative half-wave and one for the positive half-wave, the half-waves being integrated in a differential circuit before being sampled, this original circuitry introducing a specific nonlinear behaviour, the precision rectifier, together with the link sequence, introducing at a start of each new packet an equal amplitude modulation that is effective and readily recognizable, and at the same time the circuit, maintaining the average current values set manually over a wide range of load impedances, without causing alterations of modulations and of geometries used for constructing synthetic non-pain information, eliminating a troublesome sensation perceived by a patient in the step of regulation due to a temporary spurious stimulation of the A-Delta fibers.

32. The method of claim 30, wherein the last processing of the feedback signal is performed always through a programmed sampling, the sampling envisaging stabilization of the response of the circuit, via further synchronization with the real-time synthesis of the waveforms generated at the moment by the synthesizer module.

33. A primitive waveform for generating an electrical signal to be used in a therapy for the suppression of a pain, the primitive waveform being represented by one of the following vectors of values of amplitude, expressed in the hexadecimal system:
VO=B6 FE FE FE FE FE FE FE FE FE FE FE FE FE FE FE EC BY C8 B6 A4 92 7F 00 20 40 60 6E 80 80 80 80 80 80 80 80 80 80 80 80 80 80 80 80 80 80 80 80 80 80 80 80 80 80 80 80 80 80 80 80 80 80 80 80
V1=81 B6 FE FE FE FE FE FE FE FE FE FE FE FE FE FE FE FE FE FE FE FE FA EC DE DO C2 B4 A6 9A 8E 00 20 40 60 6E 80 80 80 80 80 80 80 80 80 80 80 80 80 80 80 80 80 80 80 80 80 80 80 80 80 80 80 80
V2=81 AA D4 FE FE FE FE FE FE FE FE FE FE FE FE FE FE FE FE FE FE FE FE FE FE FE FO E2 D4 C6 B8 AA 9C 8E 80 00 20 40 60 6E 80 80 80 80 80 80 80 80 80 80 80 80 80 80 80 80 80 80 80 80 80 80 80 80
V3=81 AA D4 FE FE FE FE FE FE FE FE FE FE FE FE FE FE FE FE FE FE FE FE FE FE FE FE FE F5 EC E3 BY D1 C8 BF B6 AD A5 9B 92 80 00 20 40 60 6E 80 80 80 80 80 80 80 80 80 80 80 80 80 80 80 80 80 80
V4=B6 FE FE FE FE FE FE FE FE FE FE FE FE FE FE FE EC BY C8 B6 A4 92 80 00 10 20 30 40 60 70 80 80 80 80 80 80 80 80 80 80 80 80 80 80 80 80 80 80 80 80 80 80 80 80 80 80 80 80 80 80 80 80 80 80
V5=81 B6 FE FE FE FE FE FE FE FE FE FE FE FE FE FE FE FE FE FE FE FE FA EC DE DO C2 B4 A6 9A 8E 00 10 20 30 40 60 70 80 80 80 80 80 80 80 80 80 80 80 80 80 80 80 80 80 80 80 80 80 80 80 80 80 80
V6=81 AA D4 FE FE FE FE FE FE FE FE FE FE FE FE FE FE FE FE FE FE FE FE FE FE FE FO E2 D4 C6 B8 AA 9C 8E 80 00 10 20 30 40 60 70 80 80 80 80 80 80 80 80 80 80 80 80 80 80 80 80 80 80 80 80 80 80
V7=81 AA D4 FE FE FE FE FE FE FE FE FE FE FE FE FE FE FE FE FE FE FE FE FE FE FE FE FE F5 EC E3 BY D1 C8 BF B6 AD A5 9B 92 80 00 10 20 30 40 60 70 80 80 80 80 80 80 80 80 80 80 80 80 80 80 80 80
V8=B6 FE FE FE FE FE FE FE FE FE FE FE FE FE FE FE EC BY C8 B6 A4 92 80 00 04 08 OC 10 16 1C 22 28 2E 34 3A 40 50 60 70 78 80 80 80 80 80 80 80 80 80 80 80 80 80 80 80 80 80 80 80 80 80 80 80 80
V9=81 B6 FE FE FE FE FE FE FE FE FE FE FE FE FE FE FE FE FE FE FE FE FA EC DE DO C2 B4 A6 9A 8E 00 04 08 OC 10 16 1C 22 28 2E 34 3A 40 50 60 70 78 80 80 80 80 80 80 80 80 80 80 80 80 80 80 80 80
V10=81 AA D4 FE FE FE FE FE FE FE FE FE FE FE FE FE FE FE FE FE FE FE FE FE FE FE FO E2 D4 C6 B8 AA 9C 8E 80 00 04 08 0C 10 16 1C 22 28 2E 34 3A 40 50 60 70 78 80 80 80 80 80 80 80 80 80 80 80 80
V11=81 AA D4 FE FE FE FE FE FE FE FE FE FE FE FE FE FE FE FE FE FE FE FE FE FE FE FE FE F5 EC E3 BY D1 C8 BF B6 AD A5 9B 92 80 00 04 08 OC 10 16 1C 22 28 2E 34 3A 40 50 60 70 78 80 80 80 80 80 80
V12=B6 FE FE FE FE FE FE FE FE FE FE FE FE FE FE FE EC BY C8 B6 A4 92 89 00 05 09 OE 18 IE 20 22 28 2E 34 3A 40 49 52 5B 64 6D 77 7F 80 80 80 80 80 80 80 80 80 80 80 80 80 80 80 80 80 80 80 80 80
V13=81 B6 FE FE FE FE FE FE FE FE FE FE FE FE FE FE FE FE FE FE FE FE FA EC DE DO C2 B4 A6 9A 8E 00 05 09 OE 18 IE 20 22 28 2E 34 3A 40 49 52 5B 64 6D 77 7F 80 80 80 80 80 80 80 80 80 80 80 80 80
V14=81 AA D4 FE FE FE FE FE FE FE FE FE FE FE FE FE FE FE FE FE FE FE FE FE FE FE FO E2 D4 C6 B8 AA 9C 8E 80 00 05 09 OE 18 IE 20 22 28 2E 34 3A 40 49 52 5B 64 6D 77 7F 80 80 80 80 80 80 80 80 80
V15=81 AA D4 FE FE FE FE FE FE FE FE FE FE FE FE FE FE FE FE FE FE FE FE FE FE FE FE FE F5 EC E3 BY D1 C8 BF B6 AD A5 9B 92 80 00 05 09 OE 18 IE 20 22 28 2E 34 3A 40 49 52 5B 64 6D 77 7F 80 80 80
V16=B6 FE FE FE FE FE FE FE FE FE FE FE FE FE FE FE EC BY C8 B6 A4 92 81 00 11 23 34 3F 52 59 61 63 65 67 69 6B 70 75 7B 7D 80 80 80 80 80 80 80 80 80 80 80 80 80 80 80 80 80 80 80 80 80 80 80 80
V17=81 B6 FE FE FE FE FE FE FE FE FE FE FE FE FE FE FE FE FE FE FE FE FA EC DE DO C2 B4 A6 9A 8E 00 11 23 34 3F 52 59 61 63 65 67 69 6B 70 75 7B 7D 80 80 80 80 80 80 80 80 80 80 80 80 80 80 80 80
V18=60 AA D4 FE FE FE FE FE FE FE FE FE FE FE FE FE FE FE FE FE FE FE FE FE FE FE FO E2 D4 C6 B8 AA 9C 8E 81 00 11 23 34 3F 52 59 61 63 65 67 69 6B 70 75 7B 7D 80 80 80 80 80 80 80 80 80 80 80 80.

34. A primitive waveform stored in a storage medium, for generating an electrical signal to be used in a therapy for the suppression of a pain, the primitive waveform being represented by one of the following vectors of values of amplitude, expressed in the hexadecimal system:
VO=B6 FE FE FE FE FE FE FE FE FE FE FE FE FE FE FE EC BY C8 B6 A4 92 7F 00 20 40 60 6E 80 80 80 80 80 80 80 80 80 80 80 80 80 80 80 80 80 80 80 80 80 80 80 80 80 80 80 80 80 80 80 80 80 80 80 80
V1=81 B6 FE FE FE FE FE FE FE FE FE FE FE FE FE FE FE FE FE FE FE FE FA EC DE DO C2 B4 A6 9A 8E 00 20 40 60 6E 80 80 80 80 80 80 80 80 80 80 80 80 80 80 80 80 80 80 80 80 80 80 80 80 80 80 80 80
V2=81 AA D4 FE FE FE FE FE FE FE FE FE FE FE FE FE FE FE FE FE FE FE FE FE FE FE F0 E2 D4 C6 B8 AA 9C 8E 80 00 20 40 60 6E 80 80 80 80 80 80 80 80 80 80 80 80 80 80 80 80 80 80 80 80 80 80 80 80
V3=81 AA D4 FE FE FE FE FE FE FE FE FE FE FE FE FE FE FE FE FE FE FE FE FE FE FE FE FE F5 EC E3 BY D1 C8 BF B6 AD A5 9B 92 80 00 20 40 60 6E 80 80 80 80 80 80 80 80 80 80 80 80 80 80 80 80 80 80
V4=B6 FE FE FE FE FE FE FE FE FE FE FE FE FE FE FE EC BY C8 B6 A4 92 80 00 10 20 30 40 60 70 80 80 80 80 80 80 80 80 80 80 80 80 80 80 80 80 80 80 80 80 80 80 80 80 80 80 80 80 80 80 80 80 80 80
V5=81 B6 FE FE FE FE FE FE FE FE FE FE FE FE FE FE FE FE FE FE FE FE FA EC DE DO C2 B4 A6 9A 8E 00 10 20 30 40 60 70 80 80 80 80 80 80 80 80 80 80 80 80 80 80 80 80 80 80 80 80 80 80 80 80 80 80
V6=81 AA D4 FE FE FE FE FE FE FE FE FE FE FE FE FE FE FE FE FE FE FE FE FE FE FE F0 E2 D4 C6 B8 AA 9C 8E 80 00 10 20 30 40 60 70 80 80 80 80 80 80 80 80 80 80 80 80 80 80 80 80 80 80 80 80 80 80
V7=81 AA D4 FE FE FE FE FE FE FE FE FE FE FE FE FE FE FE FE FE FE FE FE FE FE FE FE FE F5 EC E3 BY D1 C8 BF B6 AD A5 9B 92 80 00 10 20 30 40 60 70 80 80 80 80 80 80 80 80 80 80 80 80 80 80 80 80
V8=B6 FE FE FE FE FE FE FE FE FE FE FE FE FE FE FE EC BY C8 B6 A4 92 80 00 04 08 OC 10 16 1C 22 28 2E 34 3A 40 50 60 70 78 80 80 80 80 80 80 80 80 80 80 80 80 80 80 80 80 80 80 80 80 80 80 80 80
V9=81 B6 FE FE FE FE FE FE FE FE FE FE FE FE FE FE FE FE FE FE FE FE FA EC DE DO C2 B4 A6 9A 8E 00 04 08 OC 10 16 1C 22 28 2E 34 3A 40 50 60 70 78 80 80 80 80 80 80 80 80 80 80 80 80 80 80 80 80
V10=81 AA D4 FE FE FE FE FE FE FE FE FE FE FE FE FE FE FE FE FE FE FE FE FE FE FE FO E2 D4 C6 B8 AA 9C 8E 80 00 04 08 OC 10 16 1C 22 28 2E 34 3A 40 50 60 70 78 80 80 80 80 80 80 80 80 80 80 80 80
V11=81 AA D4 FE FE FE FE FE FE FE FE FE FE FE FE FE FE FE FE FE FE FE FE FE FE FE FE FE F5 EC E3 BY D1 C8 BF B6 AD A5 9B 92 80 00 04 08 OC 10 16 1C 22 28 2E 34 3A 40 50 60 70 78 80 80 80 80 80 80
V12=B6 FE FE FE FE FE FE FE FE FE FE FE FE FE FE FE EC BY C8 B6 A4 92 89 00 05 09 OE 18 IE 20 22 28 2E 34 3A 40 49 52 5B 64 6D 77 7F 80 80 80 80 80 80 80 80 80 80 80 80 80 80 80 80 80 80 80 80 80
V13=81 B6 FE FE FE FE FE FE FE FE FE FE FE FE FE FE FE FE FE FE FE FE FA EC DE DO C2 B4 A6 9A 8E 00 05 09 OE 18 IE 20 22 28 2E 34 3A 40 49 52 5B 64 6D 77 7F 80 80 80 80 80 80 80 80 80 80 80 80 80
V14=81 AA D4 FE FE FE FE FE FE FE FE FE FE FE FE FE FE FE FE FE FE FE FE FE FE FE FO E2 D4 C6 B8 AA 9C 8E 80 00 05 09 OE 18 IE 20 22 28 2E 34 3A 40 49 52 5B 64 6D 77 7F 80 80 80 80 80 80 80 80 80
V15=81 AA D4 FE FE FE FE FE FE FE FE FE FE FE FE FE FE FE FE FE FE FE FE FE FE FE FE FE F5 EC E3 BY D1 C8 BF B6 AD A5 9B 92 80 00 05 09 0E 18 IE 20 22 28 2E 34 3A 40 49 52 5B 64 6D 77 7F 80 80 80
V16=B6 FE FE FE FE FE FE FE FE FE FE FE FE FE FE FE EC BY C8 B6 A4 92 81 00 11 23 34 3F 52 59 61 63 65 67 69 6B 70 75 7B 7D 80 80 80 80 80 80 80 80 80 80 80 80 80 80 80 80 80 80 80 80 80 80 80 80
V17=81 B6 FE FE FE FE FE FE FE FE FE FE FE FE FE FE FE FE FE FE FE FE FA EC DE DO C2 B4 A6 9A 8E 00 11 23 34 3F 52 59 61 63 65 67 69 6B 70 75 7B 7D 80 80 80 80 80 80 80 80 80 80 80 80 80 80 80 80
V18=60 AA D4 FE FE FE FE FE FE FE FE FE FE FE FE FE FE FE FE FE FE FE FE FE FE FE. F0 E2 D4 C6 B8 AA 9C 8E 81 00 11 23 34 3F 52 59 61 63 65 67 69 6B 70 75 7B 7D 80 80 80 80 80 80 80 80 80 80 80 80.

35. Use of one or more primitive waveforms for generating an electrical signal to be applied in a therapy for the suppression of a pain, the electrical signal being applied to C fibers of a body so as to use the C fibers as primary vehicle for inducing analgesia, without blocking conduction of the C fibers, the electrical stimulus being transformed into “non-pain” information to obtain analgesia by exciting the C fibers.

36. The use of claim 33, wherein the one or more primitive waveforms are to be selected in a set of primitive waveforms, respectively represented by the following values of amplitude, expressed in the hexadecimal system:
VO=B6 FE FE FE FE FE FE FE FE FE FE FE FE FE FE FE EC BY C8 B6 A4 92 7F 00 20 40 60 6E 80 80 80 80 80 80 80 80 80 80 80 80 80 80 80 80 80 80 80 80 80 80 80 80 80 80 80 80 80 80 80 80 80 80 80 80
V1=81 B6 FE FE FE FE FE FE FE FE FE FE FE FE FE FE FE FE FE FE FE FE FA EC DE DO C2 B4 A6 9A 8E 00 20 40 60 6E 80 80 80 80 80 80 80 80 80 80 80 80 80 80 80 80 80 80 80 80 80 80 80 80 80 80 80 80
V2=81 AA D4 FE FE FE FE FE FE FE FE FE FE FE FE FE FE FE FE FE FE FE FE FE FE FE FO E2 D4 C6 B8 AA 9C 8E 80 00 20 40 60 6E 80 80 80 80 80 80 80 80 80 80 80 80 80 80 80 80 80 80 80 80 80 80 80 80
V3=81 AA D4 FE FE FE FE FE FE FE FE FE FE FE FE FE FE FE FE FE FE FE FE FE FE FE FE FE F5 EC E3 BY D1 C8 BF B6 AD A5 9B 92 80 00 20 40 60 6E 80 80 80 80 80 80 80 80 80 80 80 80 80 80 80 80 80 80
V4=B6 FE FE FE FE FE FE FE FE FE FE FE FE FE FE FE EC BY C8 B6 A4 92 80 00 10 20 30 40 60 70 80 80 80 80 80 80 80 80 80 80 80 80 80 80 80 80 80 80 80 80 80 80 80 80 80 80 80 80 80 80 80 80 80 80
V5=81 B6 FE FE FE FE FE FE FE FE FE FE FE FE FE FE FE FE FE FE FE FE FA EC DE DO C2 B4 A6 9A 8E 00 10 20 30 40 60 70 80 80 80 80 80 80 80 80 80 80 80 80 80 80 80 80 80 80 80 80 80 80 80 80 80 80
V6=81 AA D4 FE FE FE FE FE FE FE FE FE FE FE FE FE FE FE FE FE FE FE FE FE FE FE FO E2 D4 C6 B8 AA 9C 8E 80 00 10 20 30 40 60 70 80 80 80 80 80 80 80 80 80 80 80 80 80 80 80 80 80 80 80 80 80 80
V7=81 AA D4 FE FE FE FE FE FE FE FE FE FE FE FE FE FE FE FE FE FE FE FE FE FE FE FE FE F5 EC E3 BY D1 C8 BF B6 AD A5 9B 92 80 00 10 20 30 40 60 70 80 80 80 80 80 80 80 80 80 80 80 80 80 80 80 80
V8=B6 FE FE FE FE FE FE FE FE FE FE FE FE FE FE FE EC BY C8 B6 A4 92 80 00 04 08 OC 10 16 1C 22 28 2E 34 3A 40 50 60 70 78 80 80 80 80 80 80 80 80 80 80 80 80 80 80 80 80 80 80 80 80 80 80 80 80
V9=81 B6 FE FE FE FE FE FE FE FE FE FE FE FE FE FE FE FE FE FE FE FE FA EC DE DO C2 B4 A6 9A 8E 00 04 08 OC 10 16 1C 22 28 2E 34 3A 40 50 60 70 78 80 80 80 80 80 80 80 80 80 80 80 80 80 80 80 80
V10=81 AA D4 FE FE FE FE FE FE FE FE FE FE FE FE FE FE FE FE FE FE FE FE FE FE FE FO E2 D4 C6 B8 AA 9C 8E 80 00 04 08 OC 10 16 1C 22 28 2E 34 3A 40 50 60 70 78 80 80 80 80 80 80 80 80 80 80 80 80
V11=81 AA D4 FE FE FE FE FE FE FE FE FE FE FE FE FE FE FE FE FE FE FE FE FE FE FE FE FE F5 EC E3 BY D1 C8 BF B6 AD A5 9B 92 80 00 04 08 OC 10 16 1C 22 28 2E 34 3A 40 50 60 70 78 80 80 80 80 80 80
V12=B6 FE FE FE FE FE FE FE FE FE FE FE FE FE FE FE EC BY C8 B6 A4 92 89 00 05 09 OE 18 IE 20 22 28 2E 34 3A 40 49 52 5B 64 6D 77 7F 80 80 80 80 80 80 80 80 80 80 80 80 80 80 80 80 80 80 80 80 80
V13=81 B6 FE FE FE FE FE FE FE FE FE FE FE FE FE FE FE FE FE FE FE FE FA EC DE DO C2 B4 A6 9A 8E 00 05 09 OE 18 IE 20 22 28 2E 34 3A 40 49 52 5B 64 6D 77 7F 80 80 80 80 80 80 80 80 80 80 80 80 80
V14=81 AA D4 FE FE FE FE FE FE FE FE FE FE FE FE FE FE FE FE FE FE FE FE FE FE FE FO E2 D4 C6 B8 AA 9C 8E 80 00 05 09 OE 18 IE 20 22 28 2E 34 3A 40 49 52 5B 64 6D 77 7F 80 80 80 80 80 80 80 80 80
V15=81 AA D4 FE FE FE FE FE FE FE FE FE FE FE FE FE FE FE FE FE FE FE FE FE FE FE FE FE F5 EC E3 BY D1 C8 BF B6 AD A5 9B 92 80 00 05 09 OE 18 IE 20 22 28 2E 34 3A 40 49 52 5B 64 6D 77 7F 80 80 80
V16=B6 FE FE FE FE FE FE FE FE FE FE FE FE FE FE FE EC BY C8 B6 A4 92 81 00 11 23 34 3F 52 59 61 63 65 67 69 6B 70 75 7B 7D 80 80 80 80 80 80 80 80 80 80 80 80 80 80 80 80 80 80 80 80 80 80 80 80
V17=81 B6 FE FE FE FE FE FE FE FE FE FE FE FE FE FE FE FE FE FE FE FE FA EC DE DO C2 B4 A6 9A 8E 00 11 23 34 3F 52 59 61 63 65 67 69 6B 70 75 7B 7D 80 80 80 80 80 80 80 80 80 80 80 80 80 80 80 80
V18=60 AA D4 FE FE FE FE FE FE FE FE FE FE FE FE FE FE FE FE FE FE FE FE FE FE FE FO E2 D4 C6 B8 AA 9C 8E 81 00 11 23 34 3F 52 59 61 63 65 67 69 6B 70 75 7B 7D 80 80 80 80 80 80 80 80 80 80 80 80.

37. A method for generating an electrical signal to be used in a therapy for suppression of a pain, the electrical signal being applied to C fibers of a body so as to use them as primary vehicle for inducing analgesia, without blocking conduction of the C fibers, the electrical stimulus being transformed into “non-pain” information to obtain analgesia by exciting the C fibers, the method comprising the following steps:
supplying a set of primitive waveforms, each primitive waveform having a periodic and predetermined time plot, identified by first parameters;
calculating second parameters that can be associated to each of the primitive waveforms
processing a set of data which identify a sequence made up of one or more of the primitive waveforms in temporal sequence, each of the primitive waveforms of the sequence being processed on the basis of one or more of the second parameters; and
generating an electrical output signal corresponding to the sequence.

38. The method of claim 37, wherein the first parameters comprise values of amplitude of each primitive waveform of the set of primitive waveforms.

39. The method of claim 38, wherein each of the primitive waveforms is represented in digital format by a corresponding vector of values, expressed in the hexadecimal system:
VO=B6 FE FE FE FE FE FE FE FE FE FE FE FE FE FE FE EC BY C8 B6 A4 92 7F 00 20 40 60 6E 80 80 80 80 80 80 80 80 80 80 80 80 80 80 80 80 80 80 80 80 80 80 80 80 80 80 80 80 80 80 80 80 80 80 80 80
V1=81 B6 FE FE FE FE FE FE FE FE FE FE FE FE FE FE FE FE FE FE FE FE FA EC DE DO C2 B4 A6 9A 8E 00 20 40 60 6E 80 80 80 80 80 80 80 80 80 80 80 80 80 80 80 80 80 80 80 80 80 80 80 80 80 80 80 80
V2=81 AA D4 FE FE FE FE FE FE FE FE FE FE FE FE FE FE FE FE FE FE FE FE FE FE FE F0 E2 D4 C6 B8 AA 9C 8E 80 00 20 40 60 6E 80 80 80 80 80 80 80 80 80 80 80 80 80 80 80 80 80 80 80 80 80 80 80 80
V3=81 AA D4 FE FE FE FE FE FE FE FE FE FE FE FE FE FE FE FE FE FE FE FE FE FE FE FE FE F5 EC E3 BY D1 C8 BF B6 AD A5 9B 92 80 00 20 40 60 6E 80 80 80 80 80 80 80 80 80 80 80 80 80 80 80 80 80 80
V4=B6 FE FE FE FE FE FE FE FE FE FE FE FE FE FE FE EC BY C8 B6 A4 92 80 00 10 20 30 40 60 70 80 80 80 80 80 80 80 80 80 80 80 80 80 80 80 80 80 80 80 80 80 80 80 80 80 80 80 80 80 80 80 80 80 80
V5=81 B6 FE FE FE FE FE FE FE FE FE FE FE FE FE FE FE FE FE FE FE FE FA EC DE DO C2 B4 A6 9A 8E 00 10 20 30 40 60 70 80 80 80 80 80 80 80 80 80 80 80 80 80 80 80 80 80 80 80 80 80 80 80 80 80 80
V6=81 AA D4 FE FE FE FE FE FE FE FE FE FE FE FE FE FE FE FE FE FE FE FE FE FE FE F0E2 D4 C6 B8 AA 9C 8E 80 00 10 20 30 40 60 70 80 80 80 80 80 80 80 80 80 80 80 80 80 80 80 80 80 80 80 80 80 80
V7=81 AA D4 FE FE FE FE FE FE FE FE FE FE FE FE FE FE FE FE FE FE FE FE FE FE FE FE FE F5 EC E3 BY D1 C8 BF B6 AD A5 9B 92 80 00 10 20 30 40 60 70 80 80 80 80 80 80 80 80 80 80 80 80 80 80 80 80
V8=B6 FE FE FE FE FE FE FE FE FE FE FE FE FE FE FE EC BY C8 B6 A4 92 80 00 04 08 OC 10 16 1C 22 28 2E 34 3A 40 50 60 70 78 80 80 80 80 80 80 80 80 80 80 80 80 80 80 80 80 80 80 80 80 80 80 80 80
V9=81 B6 FE FE FE FE FE FE FE FE FE FE FE FE FE FE FE FE FE FE FE FE FA EC DE DO C2 B4 A6 9A 8E 00 04 08 OC 10 16 1C 22 28 2E 34 3A 40 50 60 70 78 80 80 80 80 80 80 80 80 80 80 80 80 80 80 80 80
V10=81 AA D4 FE FE FE FE FE FE FE FE FE FE FE FE FE FE FE FE FE FE FE FE FE FE FE FO E2 D4 C6 B8 AA 9C 8E 80 00 04 08 OC 10 16 1C 22 28 2E 34 3A 40 50 60 70 78 80 80 80 80 80 80 80 80 80 80 80 80
V11=81 AA D4 FE FE FE FE FE FE FE FE FE FE FE FE FE FE FE FE FE FE FE FE FE FE FE FE FE F5 EC E3 BY D1 C8 BF B6 AD A5 9B 92 80 00 04 08 OC 10 16 1C 22 28 2E 34 3A 40 50 60 70 78 80 80 80 80 80 80
V12=B6 FE FE FE FE FE FE FE FE FE FE FE FE FE FE FE EC BY C8 B6 A4 92 89 00 05 09 OE 18 IE 20 22 28 2E 34 3A 40 49 52 5B 64 6D 77 7F 80 80 80 80 80 80 80 80 80 80 80 80 80 80 80 80 80 80 80 80 80
V13=81 B6 FE FE FE FE FE FE FE FE FE FE FE FE FE FE FE FE FE FE FE FE FA EC DE DO C2 B4 A6 9A 8E 00 05 09 OE 18 IE 20 22 28 2E 34 3A 40 49 52 5B 64 6D 77 7F 80 80 80 80 80 80 80 80 80 80 80 80 80
V14=81 AA D4 FE FE FE FE FE FE FE FE FE FE FE FE FE FE FE FE FE FE FE FE FE FE FE FO E2 D4 C6 B8 AA 9C 8E 80 00 05 09 OE 18 IE 20 22 28 2E 34 3A 40 49 52 5B 64 6D 77 7F 80 80 80 80 80 80 80 80 80
V15=81 AA D4 FE FE FE FE FE FE FE FE FE FE FE FE FE FE FE FE FE FE FE FE FE FE FE FE FE F5 EC E3 BY D1 C8 BF B6 AD A5 9B 92 80 00 05 09 OE 18 IE 20 22 28 2E 34 3A 40 49 52 5B 64 6D 77 7F 80 80 80
V16=B6 FE FE FE FE FE FE FE FE FE FE FE FE FE FE FE EC BY C8 B6 A4 92 81 00 11 23 34 3F 52 59 61 63 65 67 69 6B 70 75 7B 7D 80 80 80 80 80 80 80 80 80 80 80 80 80 80 80 80 80 80 80 80 80 80 80 80
V17=81 B6 FE FE FE FE FE FE FE FE FE FE FE FE FE FE FE FE FE FE FE FE FA EC DE DO C2 B4 A6 9A 8E 00 11 23 34 3F 52 59 61 63 65 67 69 6B 70 75 7B 7D 80 80 80 80 80 80 80 80 80 80 80 80 80 80 30 80
V18=60 AA D4 FE FE FE FE FE FE FE FE FE FE FE FE FE FE FE FE FE FE FE FE FE FE FE FO E2 D4 C6 B8 AA 9C 8E 81 00 11 23 34 3F 52 59 61 63 65 67 69 6B 70 75 7B 7D 80 80 80 80 80 80 80 80 80 80 80 80.

40. The method of claim 37, wherein each datum of the set of data is in digital format and comprises at least:
a first portion, identifying the primitive waveform selected to be repeated in succession in the sequence, to form a packet;
a second portion, identifying a frequency value to be associated to the primitive waveform selected in the sequence; and
a third portion, identifying a first value of temporal duration to be associated to a pause interval, subsequent to the packet.

41. The method of claim 40, further comprising the step of calculating a second value of temporal duration to be associated to the duration of the packet.

42. The method of claim 37, wherein the selection of the waveforms with which to compose the sequence is executed on the basis of a first criterion of a probabilistic type.

43. The method of claim 42, wherein the first probabilistic criterion involves the variable and dynamic selection of the waveforms.

44. The method of claim 42, wherein the first probabilistic criterion is dynamically modified according to a first probabilistic filter based upon pre-set rules, in such a way as to vary in a foreseeable and organized manner a probability of selection of each of the waveforms.

45. The method of claim 37, wherein the calculation of the second parameters for each waveform included in the sequence is executed on the basis of further probabilistic criteria, starting from pre-set values.

46. The method of claim 45, wherein the further probabilistic criteria for calculating the second parameters are dynamically modified according to respective further probabilistic filters based upon corresponding pre-set rules in such a way as to vary the probability of selection of the pre-set values.

47. The method of claim 44, wherein the probabilistic filters are such as to minimize the probability of selection in succession of one and the same primitive waveform, in association with one and the same parameter of the set of parameters.

48. The method of claim 37, wherein the step of generating an output signal corresponding to the sequence comprises a step of digital-to-analog conversion of the set of data identifying the sequence.

49. The method of claim 48, further comprising the step of amplitude modulation of the output signal.
